SEQUENCE LISTING (88 sequences)

SEQ ID NO: 1 - protein, 635 aa, Homo sapiens
  REGION 1..635: note = Thrombopoietin receptor (TPOR)
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 2 - protein, 81 aa, Homo sapiens
  REGION 1..81: note = TPOR amino acids 37-117
PLKCFSRTFEDLTCFWDEEEAAPSGTYQLLYAYPREKPRACPLSSQSMPHFGTRYVCQFPDQEEVRLFFPLHLWVKNVFLN

SEQ ID NO: 3 - protein, 85 aa, Homo sapiens
  REGION 1..85: note = TPOR amino acids 37-121
PLKCFSRTFEDLTCFWDEEEAAPSGTYQLLYAYPREKPRACPLSSQSMPHFGTRYVCQFPDQEEVRLFFPLHLWVKNVFLNQTRT

SEQ ID NO: 4 - protein, 96 aa, Homo sapiens
  REGION 1..96: note = TPOR amino acids 26-121 (D1)
QDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRT

SEQ ID NO: 5 - protein, 258 aa, Homo sapiens
  REGION 1..258: note = TPOR amino acids 26-283 (D1D2)
QDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGD

SEQ ID NO: 6 - protein, 465 aa, Homo sapiens
  REGION 1..465: note = TPOR amino acids 26-490 (D1D2D3D4)
QDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETA

SEQ ID NO: 7 - protein, 19 aa, Homo sapiens
  REGION 1..19: note = TPOR amino acids 37-55
PLKCFSRTFEDLTCFWDEE

SEQ ID NO: 8 - protein, 417 aa, Homo sapiens
  REGION 1..417: note = Calreticulin (CALR)
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 9 - protein, 43 aa, Homo sapiens
  REGION 1..43: note = CALR mutant C-ter tail common amino acids
RMMRTKMRMRRMRRTRRKMRRKMSPARPRTSCREACLQGWTEA

SEQ ID NO: 10 - protein, 45 aa, Homo sapiens
  REGION 1..45: note = CALR mutant C-ter tail type 1
TRRMMRTKMRMRRMRRTRRKMRRKMSPARPRTSCREACLQGWTEA

SEQ ID NO: 11 - protein, 46 aa, Homo sapiens
  REGION 1..46: note = CALR mutant C-ter tail type 2
NCRRMMRTKMRMRRMRRTRRKMRRKMSPARPRTSCREACLQGWTEA

SEQ ID NO: 12 - protein, 47 aa, Homo sapiens
  REGION 1..47: note = CALR mutant C-ter tail type 3
QRTRRMMRTKMRMRRMRRTRRKMRRKMSPARPRTSCREACLQGWTEA

SEQ ID NO: 13 - protein, 50 aa, Homo sapiens
  REGION 1..50: note = CALR mutant C-ter tail type 4
RRRQRTRRMMRTKMRMRRMRRTRRKMRRKMSPARPRTSCREACLQGWTEA

SEQ ID NO: 14 - protein, 48 aa, Homo sapiens
  REGION 1..48: note = CALR mutant C-ter tail type 5
GQRTRRMMRTKMRMRRMRRTRRKMRRKMSPARPRTSCREACLQGWTEA

SEQ ID NO: 15 - protein, 49 aa, Homo sapiens
  REGION 1..49: note = CALR mutant C-ter tail type 6
RRQRTRRMMRTKMRMRRMRRTRRKMRRKMSPARPRTSCREACLQGWTEA

SEQ ID NO: 16 - protein, 44 aa, Homo sapiens
  REGION 1..44: note = CALR mutant C-ter tail type 7
RRMMRTKMRMRRMRRTRRKMRRKMSPARPRTSCREACLQGWTEA

SEQ ID NO: 17 - protein, 49 aa, Homo sapiens; identical to SEQ ID NO: 15
  REGION 1..49: note = CALR mutant C-ter tail type 8

SEQ ID NO: 18 - protein, 48 aa, Homo sapiens
  REGION 1..48: note = CALR mutant C-ter tail type 9
RQRTRRMMRTKMRMRRMRRTRRKMRRKMSPARPRTSCREACLQGWTEA

SEQ ID NO: 19 - protein, 46 aa, Homo sapiens
  REGION 1..46: note = CALR mutant C-ter tail type 10
MCRRMMRTKMRMRRMRRTRRKMRRKMSPARPRTSCREACLQGWTEA

SEQ ID NO: 20 - protein, 50 aa, Homo sapiens
  REGION 1..50: note = CALR mutant C-ter tail type 11
DQRQRTRRMMRTKMRMRRMRRTRRKMRRKMSPARPRTSCREACLQGWTEA

SEQ ID NO: 21 - protein, 51 aa, Homo sapiens
  REGION 1..51: note = CALR mutant C-ter tail type 12
RRRRQRTRRMMRTKMRMRRMRRTRRKMRRKMSPARPRTSCREACLQGWTEA

SEQ ID NO: 22 - protein, 51 aa, Homo sapiens
  REGION 1..51: note = CALR mutant C-ter tail type 13
QRRRQRTRRMMRTKMRMRRMRRTRRKMRRKMSPARPRTSCREACLQGWTEA

SEQ ID NO: 23 - protein, 50 aa, Homo sapiens; identical to SEQ ID NO: 13
  REGION 1..50: note = CALR mutant C-ter tail type 14

SEQ ID NO: 24 - protein, 50 aa, Homo sapiens
  REGION 1..50: note = CALR mutant C-ter tail type 15
RRRERTRRMMRTKMRMRRMRRTRRKMRRKMSPARPRTSCREACLQGWTEA

SEQ ID NO: 25 - protein, 50 aa, Homo sapiens
  REGION 1..50: note = CALR mutant C-ter tail type 16
QRRQRTRRMMRTKMRMRRMRRTRRKMRRKMSPARPRTSCREACLQGWTEA

SEQ ID NO: 26 - protein, 49 aa, Homo sapiens
  REGION 1..49: note = CALR mutant C-ter tail type 17
RRQWTRRMMRTKMRMRRMRRTRRKMRRKMSPARPRTSCREACLQGWTEA

SEQ ID NO: 27 - protein, 43 aa, Homo sapiens; identical to SEQ ID NO: 9
  REGION 1..43: note = CALR mutant C-ter tail type 18

SEQ ID NO: 28 - protein, 48 aa, Homo sapiens; identical to SEQ ID NO: 18
  REGION 1..48: note = CALR mutant C-ter tail type 19

SEQ ID NO: 29 - protein, 50 aa, Homo sapiens
  REGION 1..50: note = CALR mutant C-ter tail type 20
GRRQRTRRMMRTKMRMRRMRRTRRKMRRKMSPARPRTSCREACLQGWTEA

SEQ ID NO: 30 - protein, 49 aa, Homo sapiens
  REGION 1..49: note = CALR mutant C-ter tail type 21
AFKRTRRMMRTKMRMRRMRRTRRKMRRKMSPARPRTSCREACLQGWTEA

SEQ ID NO: 31 - protein, 54 aa, Homo sapiens
  REGION 1..54: note = CALR mutant C-ter tail type 22
NAKRRRRQRTRRMMRTKMRMRRMRRTRRKMRRKMSPARPRTSCREACLQGWTEA

SEQ ID NO: 32 - protein, 53 aa, Homo sapiens
  REGION 1..53: note = CALR mutant C-ter tail type 23
CVRRRRQRTRRMMRTKMRMRRMRRTRRKMRRKMSPARPRTSCREACLQGWTEA

SEQ ID NO: 33 - protein, 49 aa, Homo sapiens; identical to SEQ ID NO: 15
  REGION 1..49: note = CALR mutant C-ter tail type 24

SEQ ID NO: 34 - protein, 48 aa, Homo sapiens; identical to SEQ ID NO: 18
  REGION 1..48: note = CALR mutant C-ter tail type 25

SEQ ID NO: 35 - protein, 54 aa, Homo sapiens; identical to SEQ ID NO: 31
  REGION 1..54: note = CALR mutant C-ter tail type 26

SEQ ID NO: 36 - protein, 55 aa, Homo sapiens
  REGION 1..55: note = CALR mutant C-ter tail type 27
CFAKRRRRQRTRRMMRTKMRMRRMRRTRRKMRRKMSPARPRTSCREACLQGWTEA

SEQ ID NO: 37 - protein, 44 aa, Homo sapiens; identical to SEQ ID NO: 16
  REGION 1..44: note = CALR mutant C-ter tail type 28

SEQ ID NO: 38 - protein, 49 aa, Homo sapiens
  REGION 1..49: note = CALR mutant C-ter tail type 29
PPLCLRRMMRTKMRMRRMRRTRRKMRRKMSPARPRTSCREACLQGWTEA

SEQ ID NO: 39 - protein, 48 aa, Homo sapiens
  REGION 1..48: note = CALR mutant C-ter tail type 30
DHPCRRMMRTKMRMRRMRRTRRKMRRKMSPARPRTSCREACLQGWTEA

SEQ ID NO: 40 - protein, 47 aa, Homo sapiens
  REGION 1..47: note = CALR mutant C-ter tail type 31
GNCRRMMRTKMRMRRMRRTRRKMRRKMSPARPRTSCREACLQGWTEA

SEQ ID NO: 41 - protein, 45 aa, Homo sapiens
  REGION 1..45: note = CALR mutant C-ter tail type 32
CRRMMRTKMRMRRMRRTRRKMRRKMSPARPRTSCREACLQGWTEA

SEQ ID NO: 42 - protein, 45 aa, Homo sapiens; identical to SEQ ID NO: 41
  REGION 1..45: note = CALR mutant C-ter tail type 33

SEQ ID NO: 43 - protein, 46 aa, Homo sapiens
  REGION 1..46: note = CALR mutant C-ter tail type 34
TCRRMMRTKMRMRRMRRTRRKMRRKMSPARPRTSCREACLQGWTEA

SEQ ID NO: 44 - protein, 46 aa, Homo sapiens
  REGION 1..46: note = CALR mutant C-ter tail type 35
ICRRMMRTKMRMRRMRRTRRKMRRKMSPARPRTSCREACLQGWTEA

SEQ ID NO: 45 - protein, 45 aa, Homo sapiens; identical to SEQ ID NO: 41
  REGION 1..45: note = CALR mutant C-ter tail type 36

SEQ ID NO: 46 - protein, 181 aa, Homo sapiens
  REGION 1..181: note = CALR amino acids 16-197 (N domain)
AEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLE

SEQ ID NO: 47 - protein, 111 aa, Homo sapiens
  REGION 1..111: note = CALR amino acids 198-308 (P domain)
DDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAY

SEQ ID NO: 48 - protein, 109 aa, Homo sapiens
  REGION 1..109: note = CALR amino acids 309-417 (C domain)
DNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 49 - protein, 411 aa, Homo sapiens
  REGION 1..411: note = CALR del52
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 50 - protein, 51 aa, Homo sapiens
  REGION 1..51: note = CALR del52 C-ter tail
QDEEQRTRRMMRTKMRMRRMRRTRRKMRRKMSPARPRTSCREACLQGWTEA

SEQ ID NO: 51 - protein, 13 aa, Homo sapiens
  REGION 1..13: note = CALR del52 amino acids 342-354
FGNETWGVTKAAE

SEQ ID NO: 52 - protein, 26 aa, Homo sapiens
  REGION 1..26: note = CALR del52 amino acids 350-375
TKAAEKQMKDKQDEEQRTRRMMRTKM

SEQ ID NO: 53 - protein, 6 aa, Homo sapiens
  REGION 1..6: note = CALR del52 amino acids 406-411
QWGTEA

SEQ ID NO: 54 - protein, 25 aa, Homo sapiens
  REGION 1..25: note = CALR del52 amino acids 50-74
VLSSGKFYGDEEKDKGLQTSQDARF

SEQ ID NO: 55 - protein, 14 aa, Homo sapiens
  REGION 1..14: note = CALR del52 amino acids 96-109
TVKHEQNIDCGGGY

SEQ ID NO: 56 - protein, 29 aa, Homo sapiens
  REGION 1..29: note = CALR del52 amino acids 347-375
WGVTKAAEKQMKDKQDEEQRTRRMMRTKM

SEQ ID NO: 57 - protein, 8 aa, Homo sapiens
  REGION 1..8: note = TPOR amino acids 16-23
FSRTFEDL

SEQ ID NO: 58 - protein, 20 aa, Homo sapiens
  REGION 1..20: note = TPOR amino acids 37-56
PLKCFSRTFEDLTCFWDEEE

SEQ ID NO: 59 - protein, 7 aa, Homo sapiens
  REGION 1..7: note = TPOR amino acids 41-47
FSRTFED

SEQ ID NO: 60 - protein, 4 aa, Homo sapiens
  REGION 1..4: note = TPOR amino acids 44-47
TFED

SEQ ID NO: 61 - protein, 11 aa, Homo sapiens
  REGION 1..11: note = TPOR amino acids 51-62
WDEEEAAPGST

SEQ ID NO: 62 - protein, 4 aa, Homo sapiens
  REGION 1..4: note = TPOR amino acids 51-54
WDEE

SEQ ID NO: 63 - protein, 7 aa, Homo sapiens
  REGION 1..7: note = TPOR amino acids 55-62
EAAPGST

SEQ ID NO: 64 - protein, 6 aa, Homo sapiens
  REGION 1..6: note = TPOR amino acids 272-277
WSLPVT

SEQ ID NO: 65 - protein, 4 aa, Homo sapiens
  REGION 1..4: note = TPOR amino acids 64-68
QLLY

SEQ ID NO: 66 - protein, 66 aa, synthetic construct
  VARIANT 7: note = X = R or K
  VARIANT 10: note = X = D or E
  VARIANT 11: note = X = D or E
  VARIANT 17: note = X = D or E
  VARIANT 18: note = X = D or E
  VARIANT 19: note = X = D or E
  VARIANT 20: note = X = D or E
  VARIANT 61: note = X = D or E
  VARIANT 63: note = X = D or E
  VARIANT 64: note = X = D or E
  VARIANT 66: note = X = R or K
PLKCFSXTFXXLTCFWXXXXAAPSGTYQLLYAYPREKPRACPLSSQSMPHFGTRYVCQFPXQXXVX

SEQ ID NO: 67 - protein, 66 aa, Homo sapiens
PLKCFSRTFEDLTCFWDEEEAAPSGTYQLLYAYPREKPRACPLSSQSMPHFGTRYVCQFPDQEEVR

SEQ ID NO: 68 - protein, 77 aa, synthetic construct
  VARIANT 2: note = X = D or E
  VARIANT 9: note = X = D or E
  VARIANT 11: note = X = D or E
  VARIANT 18: note = X = R or K
  VARIANT 21: note = X = D or E
  VARIANT 22: note = X = D or E
  VARIANT 28: note = X = D or E
  VARIANT 29: note = X = D or E
  VARIANT 30: note = X = D or E
  VARIANT 31: note = X = D or E
  VARIANT 72: note = X = D or E
  VARIANT 74: note = X = D or E
  VARIANT 75: note = X = D or E
  VARIANT 77: note = X = R or K
QXVSLLASXSXPLKCFSXTFXXLTCFWXXXXAAPSGTYQLLYAYPREKPRACPLSSQSMPHFGTRYVCQFPXQXXVX

SEQ ID NO: 69 - protein, 77 aa, Homo sapiens
QDVSLLASDSEPLKCFSRTFEDLTCFWDEEEAAPSGTYQLLYAYPREKPRACPLSSQSMPHFGTRYVCQFPDQEEVR

SEQ ID NO: 70 - protein, 86 aa, synthetic construct
  VARIANT 2: note = X = D or E
  VARIANT 9: note = X = D or E
  VARIANT 11: note = X = D or E
  VARIANT 18: note = X = R or K
  VARIANT 21: note = X = D or E
  VARIANT 22: note = X = D or E
  VARIANT 28: note = X = D or E
  VARIANT 29: note = X = D or E
  VARIANT 30: note = X = D or E
  VARIANT 31: note = X = D or E
  VARIANT 72: note = X = D or E
  VARIANT 74: note = X = D or E
  VARIANT 75: note = X = D or E
  VARIANT 77: note = X = R or K
QXVSLLASXSXPLKCFSXTFXXLTCFWXXXXAAPSGTYQLLYAYPREKPRACPLSSQSMPHFGTRYVCQFPXQXXVXLFFPLHLWV

SEQ ID NO: 71 - protein, 86 aa, Homo sapiens
QDVSLLASDSEPLKCFSRTFEDLTCFWDEEEAAPSGTYQLLYAYPREKPRACPLSSQSMPHFGTRYVCQFPDQEEVRLFFPLHLWV

SEQ ID NO: 72 - skipped

SEQ ID NO: 73 - protein, 5 aa, Homo sapiens
WDEEE

SEQ ID NO: 74 - skipped

SEQ ID NO: 75 - protein, 4 aa, Homo sapiens
DQEE

SEQ ID NO: 76 - skipped

SEQ ID NO: 77 - protein, 5 aa, Homo sapiens
RTFED

SEQ ID NO: 78 - skipped

SEQ ID NO: 79 - protein, 6 aa, Homo sapiens
DQEEVR

SEQ ID NO: 80 - skipped

SEQ ID NO: 81 - protein, 4 aa, Homo sapiens
SDSE

SEQ ID NO: 82 - skipped

SEQ ID NO: 83 - protein, 8 aa, Homo sapiens
FFPLHLWV

SEQ ID NO: 84 - protein, 5 aa, Homo sapiens
PDQEE

SEQ ID NO: 85 - protein, 4 aa, Homo sapiens
WEEP

SEQ ID NO: 86 - protein, 4 aa, Homo sapiens
EAAP

SEQ ID NO: 87 - protein, 94 aa, synthetic construct
QDVSLLASDSEPLKCFSRTFEDLTCFWDEEEAAPSGTYQLLYAYPREKPRACPLSSQSMPHFGTRYVCQFPDQEEVRLAAAAHLWVKNVFLNQT

SEQ ID NO: 88 - protein, 94 aa, synthetic construct
QDVSLLASDSEPLKCFSRTFEDLTCFWDEEEAAPSGTYQLLYAYPREKPRACPLSSQSMPHFGTRYVCQFPDQEEVRLFFPLAAAAKNVFLNQT

(19) United States
(12) Patent Application Publication
(10) Pub. No.: US 2025/0250320 A1
(43) Pub. Date: Aug. 7, 2025

(54) THROMBOPOIETIN RECEPTOR FRAGMENTS AND USES THEREOF

(71) Applicant: UNIVERSITE CATHOLIQUE DE LOUVAIN, Louvain la Neuve (BE)

(72) Inventors: Stefan CONSTANTINESCU, Bruxelles (BE); Audrey NEDELEC, Woluwe-Saint-Lambert (BE); Nicolas PAPADOPOULOS, Bruxelles (BE)

(73) Assignee: UNIVERSITE CATHOLIQUE DE LOUVAIN, Louvain la Neuve (BE)

(21) Appl. No.: 18/853,177

(22) PCT Filed: Mar. 31, 2023
(86) PCT No.: PCT/EP2023/058572

§ 371 Date: Oct. 1, 2024

(30) Foreign Application Priority Data
Apr. 1, 2022 (EP) 22166302.4

(51) Int. Cl. C07K 14/71; A61K 38/00; A61P 7/02

(52) U.S. Cl. CPC C07K 14/71; A61P 7/02; A61K 38/00; C07K 2319/30

(57) ABSTRACT

A polypeptide including fragments of the extracellular domain of thrombopoietin receptor, a fusion protein and a pharmaceutical composition including the polypeptide. Also a method for treating and/or preventing a disease in which the polypeptide, the fusion protein, a nucleic acid including a sequence coding for the polypeptide, or a vector including the nucleic acid is administered to a subject in need thereof.

### FIELD OF INVENTION

The present invention relates to a polypeptide comprising at least one fragment of the thrombopoietin receptor and its use as a medicament. The present invention also relates to the treatment and/or prevention of Myeloproliferative Neoplasms (MPNs).

### BACKGROUND OF INVENTION

Malignant blood diseases represent a significant burden for the healthcare systems. Several such conditions such as BCR-ABL negative Myeloproliferative Neoplasms (MPNs) are diseases of aged individuals, with many complications including thrombosis and bleeding necessitating major resources for treatment. MPNs can evolve to a very severe condition called secondary Acute Myeloid Leukemia (AML) which is almost always fatal. The three major MPNs types, namely Polycythemia Vera (PV), Essential Thromocythemia (ET) and Myelofibrosis (MF) can occur in children and young adults but is most common in aged individuals where incidence can reach 1/5,000 persons per year. In all three diseases acquired mutations in the hematopoietic stem cells (HSCs) lead to pathological activation of the TPOR/JAK2 signaling that drives the pathology.

The JAK2 V617F mutation is responsible for 70% of all MPNs and is present in over 96% of PV and 60% of ET and MF cases, with a 70% overall involvement in BCR-ABL-negative MPNs. The rest of ET and MF cases are mostly due to acquired mutations in calreticulin (CALR) (20-30%), a chaperone retained in the endoplasmic reticulum for the quality control of N-glycosylated proteins and for calcium storage, and or the thrombopoietin receptor (TPOR) (3-5%).

It was shown that the JAK2 V617F mutation worked by inducing dimerization of the receptor through its intracellular domain in absence of cytokine thanks to interactions between the pseudokinase domains of mutant JAK2 molecules appended to the receptor.

It was also elucidated that CALR mutants bind to TPOR to induce its activation in absence of its ligand, the thrombopoietin (THPO or TPO), leading to persistent activation of the JAK2-STAT5 pathway in HSCs and megakaryocytes (Chachoua et al., 2016; Pecquet et al., 2019). Chachoua et al., 2016 also discloses that a region of TPOR and its associated N-glycosylation site (N117) are required for CALR mutants activity. Pecquet et al., 2019 describes the FFPLHWLV motif of TPOR as important for activation, but not for binding.

Different mutations have been identified in the exon 9, the last exon of CALR. More than 60 nucleotide deletions or insertions in CALR are described in MPNs, but all of them lead to the same+1 frameshift that is responsible for the synthesis of a new C-terminus of CALR (also denoted mutant C-tail or mutant tail) which is methionine rich, positively charged and devoid of the retention KDEL motif. The new tail of CALR confers tight binding to TPOR. The TPOR-CALR mutant complex travels to the cell surface and induces constitutive activation of the JAK2/STAT5 pathway in megakaryocyte progenitors and stem cells where TPOR is expressed.

Irrespective of the driver mutation, the outcome is always an over-activation of the JAK2-STAT5 pathway via TPOR and, to a lesser extent, other cytokine receptors (EpoR with JAK2 mutants, G-CSFR with JAK2 and CALR mutants). Until now, therapeutic interventions focused on non-specific JAK2 inhibitors, but failed to provide adequate clinical response in the sense of decreasing allele burdens, blasts and fibrosis. The absolute requirement and central role of TPOR for the development of all three diseases makes it a major, yet poorly explored, target for therapeutical interventions.

The Inventors have shown that specific domains or subdomains of TPOR are involved in the interaction with CALR mutants and compete or partially block the abnormally elevated TPOR induced signaling which is critical for the development of all types of MPNs.

### SUMMARY

The present invention thus relates to a polypeptide comprising an amino acid sequence having at least 75% sequence identity with SEQ ID NO: 66, and wherein said amino acid sequence of said polypeptide does not comprise SEQ ID NO: 5.

The present invention thus relates to a polypeptide comprising an amino acid sequence having at least 75% sequence identity with SEQ ID NO: 2, and wherein said amino acid sequence of said polypeptide does not comprise SEQ ID NO: 5.

The present invention thus relates to a polypeptide comprising an amino acid sequence having at least 75% sequence identity with SEQ ID NO: 2, and wherein said amino acid sequence of said polypeptide does not comprise SEQ ID NO: 6.

In some embodiments, the amino acid sequence of the polypeptide has at least 75% sequence identity with SEQ ID NO: 2, SEQ ID NO: 3 or SEQ ID NO: 4.

In some embodiments, the amino acid sequence of the polypeptide has at least 75% sequence identity with SEQ ID NO: 3, SEQ ID NO: 4 or SEQ ID NO: 5.

In some embodiments, the amino acid sequence of the polypeptide does not comprise SEQ ID NO: 5.

In some embodiments, the amino acid sequence of the polypeptide has at least 75% sequence identity with SEQ ID NO: 3 or SEQ ID NO: 4.

In some embodiment, the amino acid sequence of the polypeptide comprises at least SEQ ID NO: 7.

In some embodiments, the polypeptide binds to mutants of calreticulin (CALR) having a positively charged amino acid sequence in the C-terminus tail.

In some embodiments, the amino acid sequence of the polypeptide comprises SEQ ID NO: 7 and/or at least an amino acid Asn at position 117, wherein said position is defined with respect to the amino acid sequence SEQ ID NO: 1.

In some embodiments, the amino acid Asn at position 117 is glycosylated.

In some embodiments, the amino acid Asn at position 117 is not glycosylated.

The present invention further relates to a fusion protein comprising the polypeptide of the invention.

In one embodiment, the fusion protein further comprises a second polypeptide that increases stability and/or decreases immunogenicity of the first polypeptide.

In one embodiment, the second polypeptide of the fusion protein is a Fc region of an immunoglobulin or a functional equivalent thereof, preferably selected from the group comprising or consisting of IgG, IgA, IgD, IgE or IgM.

The present invention further relates to a nucleic acid comprising a sequence encoding the polypeptide of the invention or fusion protein comprising said polypeptide.

The present invention further relates to a vector comprising a nucleic acid encoding the polypeptide of the invention or fusion protein comprising said polypeptide.

The present invention further relates to a pharmaceutical composition comprising (i) the polypeptide, fusion protein, nucleic acid according, or vector according to the invention, and (ii) at least one pharmaceutically acceptable vehicle.

The present invention further relates to a polypeptide, fusion protein, nucleic acid, vector or pharmaceutical composition according to the invention, for use as a medicament.

In some embodiments, the polypeptide, fusion protein, nucleic acid, vector or pharmaceutical composition according to the invention are for use in the treatment and/or prevention of myeloproliferative neoplasms (MPN).

In some embodiments, the MPN is induced by one or more mutation(s) in calreticulin (CALR), preferably resulting in the generation of a positively charged amino acid sequence in the C-terminus of CALR.

The present invention further relates to a kit for treating and/or preventing myeloproliferative neoplasms (MPN) comprising (i) a polypeptide, fusion protein, nucleic acid, vector or pharmaceutical composition for use according to the invention, (ii) means to administer said polypeptide, fusion protein or pharmaceutical composition, and optionally (iii) a further anticancer agent.

### Definitions

In the present invention, the following terms have the following meanings:

The term “about”, when preceding a figure, means plus or less 10% of the value of said figure.

The term “amino acid substitution” refers to the replacement in a polypeptide of one amino acid with another amino acid. In one embodiment, an amino acid is replaced with another amino acid having similar structural and/or chemical properties, e.g., conservative amino acid replacements. “Conservative amino acid substitution” may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, tyrosine and methionine; polar neutral amino acids include glycine, serine, threonine, cysteine, asparagine, and glutamine; positively charged (basic) amino acids include arginine, lysine, and histidine; negatively charged (acidic) amino acids include aspartic acid and glutamic acid. Non-conservative substitutions will entail exchanging a member of one of these classes for another class. For example, amino acid substitutions can also result in replacing one amino acid with another amino acid having different structural and/or chemical properties, for example, replacing an amino acid from one group (e.g., polar) with another amino acid from a different group (e.g., basic). Amino acid substitutions can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

The term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: Computational Molecular Biology, Lesk, A. M., ed., Oxford University Press, New York, 1988; Biocomputing: Informatics And Genome Projects, Smith, D. W., ed., Academic Press, New York, 1993; Computer Analysis Of Sequence Data, Part I, Griffin, A. M., and Griffin, H. G., eds, Humana Press, New Jersey, 1994; Sequence Analysis In Molecular Biology, von Heijne, G., Academic Press, 1987; and Sequence Analysis Primer, Gribskov, M. and Devereux, J., eds, M Stockton Press, New York, 1991. While there exist a number of methods to measure identity between two polynucleotide or polypeptide sequences, the term “identity” is well known to skilled artisans (Carillo and Lipton, SIAM J Applied Math, 1998, 48:1073). Methods commonly employed to determine identity or similarity between two sequences include, but are not limited to, those disclosed in Guide to Huge Computers, Martin J. Bishop, ed., Academic Press, San Diego, 1994; and Carillo and Lipton, SIAM J Applied Math, 1998, 48:1073. Methods to determine identity and similarity are codified in computer programs. Computer program methods to determine identity and similarity between two sequences include, but are not limited to, GCG program package (Devereux et al., J Molec Biol, 1990, 215:403), the GAP program. As an illustration, by a polynucleotide having a nucleotide sequence having at least, for example, 95% “identity” to a reference nucleotide sequence is intended that the nucleotide sequence of the polynucleotide is identical to the reference sequence except that the polynucleotide sequence may include an average up to five point-mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

The term “peptide linker”, or “linker” also called “spacer peptide”, refers to a peptide used to link 2 peptides or polypeptides together. In one embodiment, a peptide linker of the invention comprises from 3 to 50 amino acids. Peptide linkers are known in the art or are described herein.

The term “pharmaceutically acceptable excipient” refers to an excipient that does not produce an adverse, allergic or other untoward reaction when administered to an animal, preferably a human. It includes any and all solvents, dispersion media, coatings, antibacterial and antifungal agents, isotonic and absorption delaying agents and the like. A pharmaceutically acceptable carrier or excipient refers to a non-toxic solid, semi-solid or liquid filler, diluent, encapsulating material or formulation auxiliary of any type. For human administration, preparations should meet sterility, pyrogenicity, general safety and purity standards as required by FDA Office of Biologics standards.

The term “nucleic acid” or “polynucleotide” refers to any polyribonucleotide or polydeoxyribonucleotide, which may be unmodified RNA or DNA or modified RNA or DNA. “Nucleic acid” or “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Nucleic acid” or “polynucleotide” refers to triple-stranded regions comprising RNA or DNA or both RNA and DNA. The term “nucleic acid” or “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “nucleic acid” or “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

The term “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

The term “protein” refers to a sequence of more than 100 amino acids and/or to a multimeric entity. The proteins of the invention are not limited to a specific length of the product. The term “polypeptide” or “protein” does not refer to or exclude post-expression modifications of the protein, for example, glycosylation, acetylation, phosphorylation and the like, as well as other modifications known in the art, both naturally occurring and non-naturally occurring. Such modifications are well described in basic texts and in more detailed monographs, as well as in a voluminous research literature. Modifications can occur anywhere in a polypeptide or protein, including the peptide backbone, the amino acid side-chains and the amino or carboxyl termini. It will be appreciated that the same type of modification may be present in the same or varying degrees at several sites in a given polypeptide or protein. Also, a given polypeptide or protein may contain many types of modifications. Polypeptides or proteins may be branched as a result of ubiquitination, and they may be cyclic, with or without branching. Cyclic, branched and branched cyclic polypeptides or proteins may result from posttranslational natural processes or may be made by synthetic methods. Modifications include acetylation, acylation, ADP-ribosylation, amidation, covalent attachment of flavin, covalent attachment of a hem moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-linkings, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino of amino acids to proteins such as arginylation, and ubiquitination. See, for instance, “Proteins-structure and molecular properties”, 2nd Ed., T. E. Creighton, W. H. Freeman and Comany, New York, 1993; Wolt, F., “Posttranslational Protein Modifications: Perspectives and Prospects”, Posttranslational covalent modification of proteins, B. C. Johnson, Ed., Academic Press, New York, 1983, pgs. 1-12; Seifter et al., “Analysis for protein modifications and nonprotein cofactors”, Meth Enzymol, 1990, 182:626-646; Rattan et al, “Protein Synthesis: Posttranslational Modifications and Aging”, Ann NY Acad Sci, 1992, 663:48-62. A protein may be an entire protein, or a subsequence thereof.

The term “fusion protein” refers to a protein having at least two heterologous polypeptides covalently linked either directly or via an amino acid linker. The polypeptides forming the fusion protein are typically linked C-terminus to N-terminus, although they can also be linked C-terminus to C-terminus, N-terminus to N-terminus, or N-terminus to C-terminus. The polypeptides of the fusion proteins may be fused in any order. This term also refers to conservatively modified variants, polymorphic variants, alleles, mutants, subsequences, and interspecies homologs of the antigens that make up the fusion protein.

The term “fused” refers to components that are linked by peptide bonds, either directly or through one or more peptide linkers.

The term “immunoglobulin” includes a protein having a combination of two heavy and two light chains, whether or not it possesses any relevant specific immunoreactivity.

The term “treating” or “treatment” or “alleviation” refers to both therapeutic treatment and prophylactic or preventative measures; wherein the object is to prevent or slow down (lessen) the disease or condition, preferably blood cancer, more preferably MPN. Those in need of treatment include those already affected with the disease or condition, preferably blood cancer, more preferably MPN, as well as those prone to have the disease or condition, preferably blood cancer, more preferably MPN, or those in whom the disease or condition, preferably blood cancer, more preferably MPN, is to be prevented. A subject or mammal is successfully “treated” for the disease or condition, preferably blood cancer, more preferably MPN, if, after receiving a therapeutic amount of a polypeptide or fusion protein according to the methods of the present invention, the patient shows observable and/or measurable reduction in or absence of one or more of the following: reduction in the number of pathogenic cells; reduction in the percent of total cells that are pathogenic; and/or relief to some extent, one or more of the symptoms associated with the disease or condition, preferably blood cancer, more preferably MPN; reduced morbidity and mortality, and improvement in quality of life issues. The above parameters for assessing successful treatment and improvement in the disease or condition, preferably blood cancer, more preferably MPN, are readily measurable by routine procedures familiar to a physician.

The term “variant” refers to a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions identical. A variant and reference polypeptide may differ in amino acid sequence by one or more substitutions (preferably conservative), additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants should retain one or more of the biological activities of the reference polypeptide.

### DETAILED DESCRIPTION

Thrombopoietin receptor, herein referred to as TPOR, is a transmembrane protein mostly expressed at the plasma membrane of megakaryocytes, platelets, hemangioblasts, and hematopoietic stem cells, that triggers the JAK/STAT signaling pathway upon binding to its ligand thrombopoietin. In the present disclosure, the full amino acid sequence of TPOR is referred to as SEQ ID NO: 1 (corresponding to UniProt ID P40238).

TPOR is a 635 amino acids long protein that has three functional domains: an extracellular domain (ECD) comprising the thrombopoietin binding site, a transmembrane domain (TMD), and a cytoplasmic/intracellular domain (ICD). In particular, the ECD is divided into 4 subdomains, namely D1, D2, D3 and D4.

The present invention relates to a polypeptide comprising an amino acid sequence having at least 75% sequence identity with SEQ ID NO: 66, wherein X₁ is D (Asp, Aspartic acid) or E (Glu, Glutamic acid), wherein X₂ is R (Arg, Arginine) or K (Lys, Lysine), and wherein said amino acid sequence of the polypeptide does not comprise SEQ ID NO: 5.

SEQ ID NO: 66:
PLKCFSX₂TFX₁X₁LTCFWX₁X₁X₁X₁AAPSGTYQLLYAYPREKPRACPLS
SQSMPHFGTRYVCQFPX₁QX₁X₁VX₂

In one embodiment, the polypeptide of the invention comprises an amino acid sequence having at least 75% identical to SEQ ID NO: 66, wherein the amino acid sequence comprises at least the motifs TFX₁X₁ (SEQ ID NO: 72), WX₁X₁X₁X₁ (SEQ ID NO: 74), and X₁QX₁X₁ (SEQ ID NO: 76), wherein X₁ is D (Asp, Aspartic acid) or E (Glu, Glutamic acid). In one embodiment, the polypeptide of the invention comprises an amino acid sequence having at least 75% sequence identity with SEQ ID NO: 66, wherein the amino acid sequence comprises at least the motifs TFED (SEQ ID NO: 60), WDEEE (SEQ ID NO: 73), and DQEE (SEQ ID NO: 75).

In one embodiment, the polypeptide of the invention comprises an amino acid sequence having at least 75% sequence identity with SEQ ID NO: 66, wherein the amino acid sequence comprises at least the motifs X₂TFX₁X₁(SEQ ID NO: 78), WX₁X₁X₁X₁ (SEQ ID NO: 74), and X₁QX₁X₁VX₂ (SEQ ID NO: 80), wherein X₁ is D (Asp, Aspartic acid) or E (Glu, Glutamic acid) and wherein X₂ is R (Arg, Arginine) or K (Lys, Lysine). In one embodiment, the polypeptide of the invention comprises an amino acid sequence having at least 75% sequence identity with SEQ ID NO: 66, wherein the amino acid sequence comprises at least the motifs RTFED (SEQ ID NO: 77), WDEEE (SEQ ID NO: 73), and DQEEVR (SEQ ID NO: 79).

In one embodiment, the polypeptide of the invention comprises an amino acid sequence having at least 80% sequence identity with SEQ ID NO: 66, preferably at least 85%, 90%, 95%, 96%, 97%, 98%, 99% or 100% identical. In one embodiment, the amino acid sequence of the polypeptide of the invention comprises or consists of SEQ ID NO: 66.

In one embodiment, the polypeptide of the invention comprises an amino acid sequence having at least 75% sequence identity with SEQ ID NO: 67, wherein said amino acid sequence of the polypeptide does not comprise SEQ ID NO: 5.

SEQ ID NO: 67:
PLKCFSRTFEDLTCFWDEEEAAPSGTYQLLYAYPREKPRACPLSSQSMP
HFGTRYVCQFPDQEEVR

In one embodiment, the polypeptide of the invention comprises an amino acid sequence having at least 80% sequence identity with SEQ ID NO: 67, preferably at least 85%, 90%, 95%, 96%, 97%, 98%, 99% or 100% identical. In one embodiment, the polypeptide of the invention comprises an amino acid sequence having at least 75% sequence identity with SEQ ID NO: 67, wherein the amino acid sequence comprises at least the motifs RTFED, WDEEE, and DQEEVR. In one embodiment, the amino acid sequence of the polypeptide of the invention comprises or consists of SEQ ID NO: 67.

In one embodiment, the polypeptide of the invention comprises an amino acid sequence having at least 75% sequence identity with SEQ ID NO: 68, wherein X₁ is D (Asp, Aspartic acid) or E (Glu, Glutamic acid), wherein X₂ is R (Arg, Arginine) or K (Lys, Lysine), and wherein said amino acid sequence of the polypeptide does not comprise SEQ ID NO: 5.

SEQ ID NO: 68:
QX₁VSLLASX₁SX₁PLKCFSX₂TFX₁X₁LTCFWX₁X₁X₁X₁AAPSGTYQLLY
AYPREKPRACPLSSQSMPHFGTRYVCQFPX₁QX₁X₁VX₂

In one embodiment, the polypeptide of the invention comprises an amino acid sequence having at least 75% sequence identity with SEQ ID NO: 68, wherein the amino acid sequence comprises at least the motifs QX₁V, SX₁SXᵢ(SEQ ID NO: 82), TFX₁X₁, WX₁X₁X₁X₁, and X₁QX₁X₁, wherein X₁ is D (Asp, Aspartic acid) or E (Glu, Glutamic acid). In one embodiment, the polypeptide of the invention comprises an amino acid sequence having at least 75% sequence identity with SEQ ID NO: 68, wherein the amino acid sequence comprises at least the motifs QDV, SDSE (SEQ ID NO: 81), TFED, WDEEE, and DQEE.

In one embodiment, the polypeptide of the invention comprises an amino acid sequence having at least 75% sequence identity with SEQ ID NO: 68, wherein the amino acid sequence comprises at least the motifs QX₁V, SX₁SX₁, X₂TFX₁X₁, WX₁X₁X₁X₁, and X₁QX₁X₁VX₂, wherein X₁ is D (Asp, Aspartic acid) or E (Glu, Glutamic acid) and wherein X₂ is R (Arg, Arginine) or K (Lys, Lysine). In one embodiment, the polypeptide of the invention comprises an amino acid sequence having at least 75% sequence identity with SEQ ID NO: 68, wherein the amino acid sequence comprises at least the motifs QDV, SDSE, RTFED, WDEEE, and DQEEVR.

In one embodiment, the polypeptide of the invention comprises an amino acid sequence having at least 80% sequence identity with SEQ ID NO: 68, preferably at least 85%, 90%, 95%, 96%, 97%, 98%, 99% or 100% identical. In one embodiment, the amino acid sequence of the polypeptide of the invention comprises or consists of SEQ ID NO: 68.

In one embodiment, the polypeptide of the invention comprises an amino acid sequence having at least 75% sequence identity with SEQ ID NO: 69, wherein said amino acid sequence of the polypeptide does not comprise SEQ ID NO: 5.

SEQ ID NO: 69:
QDVSLLASDSEPLKCFSRTFEDLTCFWDEEEAAPSGTYQLLYAYPREKP
RACPLSSQSMPHFGTRYVCQFPDQEEVR

In one embodiment, the polypeptide of the invention comprises an amino acid sequence having at least 75% sequence identity with SEQ ID NO: 69, wherein the amino acid sequence comprises at least the motifs QDV, SDSE, TFED, WDEEE, and DQEE.

In one embodiment, the polypeptide of the invention comprises an amino acid sequence having at least 75% sequence identity with SEQ ID NO: 69, wherein the amino acid sequence comprises at least the motifs QDV, SDSE, RTFED, WDEEE, and DQEEVR.

In one embodiment, the polypeptide of the invention comprises an amino acid sequence having at least 80% sequence identity with SEQ ID NO: 69, preferably at least 85%, 90%, 95%, 96%, 97%, 98%, 99% or 100% identical. In one embodiment, the amino acid sequence of the polypeptide of the invention comprises or consists of SEQ ID NO: 69.

In one embodiment, the polypeptide of the invention comprises an amino acid sequence having at least 75% sequence identity with SEQ ID NO: 70, wherein X₁ is D (Asp, Aspartic acid) or E (Glu, Glutamic acid), wherein X₂ is R (Arg, Arginine) or K (Lys, Lysine), and wherein said amino acid sequence of the polypeptide does not comprise SEQ ID NO: 5.

SEQ ID NO: 70:
QX₁VSLLASX₁SX₁PLKCFSX₂TFX₁X₁LTCFWX₁X₁X₁X₁AAPSGTYQLLYA
YPREKPRACPLSSQSMPHFGTRYVCQFPX₁QX₁X₁VX₂LFFPLHLWV

In one embodiment, the polypeptide of the invention comprises an amino acid sequence having at least 80% sequence identity with SEQ ID NO: 70, preferably at least 85%, 90%, 95%, 96%, 97%, 98%, 99% or 100% identical. In one embodiment, the amino acid sequence of the polypeptide of the invention comprises or consists of SEQ ID NO: 70.

In one embodiment, the polypeptide of the invention comprises an amino acid sequence having at least 75% sequence identity with SEQ ID NO: 70, wherein the amino acid sequence comprises at least the motifs QX₁V, SX₁SX₁, TFX₁X₁, WX₁X₁X₁X₁, and X₁QX₁X₁, wherein X₁ is D (Asp, Aspartic acid) or E (Glu, Glutamic acid). In one embodiment, the polypeptide of the invention comprises an amino acid sequence having at least 75% sequence identity with SEQ ID NO: 70, wherein the amino acid sequence comprises at least the motifs QDV, SDSE, TFED, WDEEE, and DQEE.

In one embodiment, the polypeptide of the invention comprises an amino acid sequence having at least 75% sequence identity with SEQ ID NO: 70, wherein the amino acid sequence comprises at least the motifs QX₁V, SX₁SX₁, X₂TFX₁X₁, WX₁X₁X₁X₁, and X₁QX₁X₁VX₂, wherein X₁ is D (Asp, Aspartic acid) or E (Glu, Glutamic acid) and wherein X₂ is R (Arg, Arginine) or K (Lys, Lysine). In one embodiment, the polypeptide of the invention comprises an amino acid sequence having at least 75% sequence identity with SEQ ID NO: 70, wherein the amino acid sequence comprises at least the motifs QDV, SDSE, RTFED, WDEEE, and DQEEVR.

In one embodiment, the polypeptide of the invention comprises an amino acid sequence having at least 75% sequence identity with SEQ ID NO: 71, wherein said amino acid sequence of the polypeptide does not comprise SEQ ID NO: 5.

SEQ ID NO: 71:
QDVSLLASDSEPLKCFSRTFEDLTCFWDEEEAAPSGTYQLLYAYPREKP
RACPLSSQSMPHFGTRYVCQFPDQEEVRLFFPLHLWV

In one embodiment, the polypeptide of the invention comprises an amino acid sequence having at least 80% sequence identity with SEQ ID NO: 71, preferably at least 85%, 90%, 95%, 96%, 97%, 98%, 99% or 100% identical. In one embodiment, the amino acid sequence of the polypeptide of the invention comprises or consists of SEQ ID NO: 71.

In one embodiment, the polypeptide of the invention comprises an amino acid sequence having at least 75% sequence identity with SEQ ID NO: 71, wherein the amino acid sequence comprises at least the motifs QDV, SDSE, TFED, WDEEE, and DQEE.

In one embodiment, the polypeptide of the invention comprises an amino acid sequence having at least 75% sequence identity with SEQ ID NO: 71, wherein the amino acid sequence comprises at least the motifs QDV, SDSE, RTFED, WDEEE, and DQEEVR.

The present invention also relates to a polypeptide comprising an amino acid sequence having at least 75% sequence identity with SEQ ID NO: 2, wherein said amino acid sequence of the polypeptide does not comprise SEQ ID NO: 5.

The present invention also relates to a polypeptide comprising an amino acid sequence having at least 75% sequence identity with SEQ ID NO: 2, wherein said amino acid sequence of the polypeptide does not comprise SEQ ID NO: 6.

SEQ ID NO: 2 relates to the sequence of the subdomain D1 of the ECD (hereinafter referred to as D1).

SEQ ID NO: 2:
PLKCFSRTFEDLTCFWDEEEAAPSGTYQLLYAYPREKPRACPLSSQSMP
HFGTRYVCQFPDQEEVRLFFPLHLWVKNVFLN

SEQ ID NO: 6 relates to the sequence of the full extracellular domain (ECD) of wild type TPOR, and encompasses subdomains D1, D2, D3 and D4 of the ECD (hereinafter referred to as D1D2D3D4).

In one embodiment, the polypeptide of the invention is an isolated polypeptide.

In one embodiment, the amino acid sequence of the polypeptide of the invention does not comprise SEQ ID NO: 1.

In some embodiments, the polypeptide of the invention comprises at least 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 amino acids.

In some embodiments, the polypeptide of the invention comprises from 66 to 464 amino acids, from 66 to 258 amino acids, from 66 to 96 amino acids, or from 66 to 85 amino acids.

In some embodiments, the polypeptide of the invention comprises from 66 to 400 amino acids, preferably from 66 to 300 amino acids, more preferably from 66 to 200 amino acids, even more preferably from 66 to 150 amino acids. In one embodiments, the polypeptide of the invention comprises from 66 to 100 amino acids, from 66 to 95, from 66 to 90, or from 66 to 85 amino acids.

In some embodiments, the polypeptide of the invention comprises from 77 to 464 amino acids, from 77 to 258 amino acids, from 77 to 96 amino acids, or from 77 to 85 amino acids.

In some embodiments, the polypeptide of the invention comprises from 77 to 400 amino acids, preferably from 77 to 300 amino acids, more preferably from 77 to 200 amino acids, even more preferably from 77 to 150 amino acids. In one embodiments, the polypeptide of the invention comprises from 77 to 100 amino acids, from 77 to 95, from 77 to 90, or from 77 to 85 amino acids.

In some embodiments, the polypeptide of the invention comprises from 81 to 464 amino acids, from 81 to 258 amino acids, from 81 to 96 amino acids, or from 81 to 85 amino acids.

In some embodiments, the polypeptide of the invention comprises from 81 to 400 amino acids, preferably from 81 to 300 amino acids, more preferably from 81 to 200 amino acids, even more preferably from 81 to 150 amino acids. In one embodiments, the polypeptide of the invention comprises from 81 to 100 amino acids, from 81 to 95, from 81 to 90, or from 81 to 85 amino acids.

In some embodiments, the polypeptide of the invention comprises from 85 to 400 amino acids, preferably from 85 to 300 amino acids, more preferably from 85 to 200 amino acids, even more preferably from 85 to 150 amino acids. In one embodiments, the polypeptide of the invention comprises from 85 to 100 amino acids, from 85 to 95, or from 85 to 90 amino acids.

In some embodiments, the polypeptide of the invention comprises from 96 to 400 amino acids, preferably from 96 to 300 amino acids, more preferably from 96 to 200 amino acids, even more preferably from 96 to 150 amino acids or from 96 to 100 amino acids.

In some embodiments, the polypeptide of the invention comprises at most 635 amino acids. In one embodiment, the polypeptide comprises at most 465 amino acids. In another embodiment, the polypeptide comprises at most 464 amino acids.

In one embodiment, the polypeptide of the invention comprises at most 600, 500, 400, 300, or 200 amino acids. In one embodiment, the polypeptide of the invention comprises at most 190, 180, 170, 160, 150, 140, 130, 125, 120, 115, 110, 105 or 100, 95, 90 or 85 amino acids. In one embodiment, the polypeptide of the invention comprises at most 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68 or 67 amino acids.

In one embodiment, the amino acid sequence of the polypeptide of the invention is at least 80% identical to SEQ ID NO: 2, preferably at least 85%, 90%, 95%, 96%, 97%, 98%, 99% or 100% identical. In one embodiment, the amino acid sequence of the polypeptide of the invention consists of SEQ ID NO: 2.

In some embodiments, the FFPLHLWV (SEQ ID NO: 83) motif within SEQ ID NO: 2, 3, 4, 70 or 71 may be absent or mutated. As shown by the Inventors, mutation of this motif does not prevent competition between TPOR and D1 for CALR mutant binding.

In one embodiment, the polypeptide of the invention comprises an amino acid sequence having at least 75% sequence identity with SEQ ID NO: 3, preferably at least 80%, 85%, 90%, 95%, 96%, 97%, 98%, 99% or 100% identical. In one embodiment, the amino acid sequence of the polypeptide of the invention comprises SEQ ID NO: 3.

In one embodiment, the polypeptide of the invention comprises an amino acid sequence having at least 75% sequence identity with SEQ ID NO: 3, preferably at least 80%, 85%, 90%, 95%, 96%, 97%, 98%, 99% or 100% identical. In one embodiment, the amino acid sequence of the polypeptide of the invention comprises or consists of SEQ ID NO: 3.

SEQ ID NO: 3:
PLKCFSRTFEDLTCFWDEEEAAPSGTYQLLYAYPREKPRACPLSSQSMP
HFGTRYVCQFPDQEEVRLFFPLHLWVKNVFLNQTRT

In one embodiment, the polypeptide of the invention comprises an amino acid sequence having at least 75% sequence identity with SEQ ID NO: 4, preferably at least 80%, 85%, 90%, 95%, 96%, 97%, 98%, 99% or 100% identical. In one embodiment, the amino acid sequence of the polypeptide of the invention comprises or consists of SEQ ID NO: 4.

SEQ ID NO: 4:
QDVSLLASDSEPLKCFSRTFEDLTCFWDEEEAAPSGTYQLLYAYPREKP
RACPLSSQSMPHFGTRYVCQFPDQEEVRLFFPLHLWVKNVFLNQTRT

In some embodiments, the amino acid sequence of the polypeptide has at least 75% sequence identity with SEQ ID NO: 3 or SEQ ID NO: 4.

In one embodiment, the polypeptide of the invention comprises an amino acid sequence having at least 75% sequence identity with SEQ ID NO: 5, preferably at least 80%, 85%, 90%, 95%, 96%, 97%, 98%, 99% or 100% identical. In one embodiment, the amino acid sequence of the polypeptide of the invention comprises or consists of SEQ ID NO: 5.

SEQ ID NO: 5 relates to the sequence of the subdomains D1 and D2 of the ECD (hereinafter referred to as D1D2).

SEQ ID NO: 5:
QDVSLLASDSEPLKCFSRTFEDLTCFWDEEEAAPSGTYQLLYAYPREKP
RACPLSSQSMPHFGTRYVCQFPDQEEVRLFFPLHLWVKNVFLNQTRTQR
VLFVDSVGLPAPPSIIKAMGGSQPGELQISWEEPAPEISDFLRYELRYG
PRDPKNSTGPTVIQLIATETCCPALQRPHSASALDQSPCAQPTMPWQDG
PKQTSPSREASALTAEGGSCLISGLQPGNSYWLQLRSEPDGISLGGSWG
SWSLPVTVDLPGD

In some embodiments, the amino acid sequence of the polypeptide of the invention does not comprise SEQ ID NO: 5. In some embodiments, the amino acid sequence of the polypeptide of the invention does not consist of SEQ ID NO: 5.

In one embodiment, the amino acid sequence of the polypeptide of the invention comprises the amino acid sequence SEQ ID NO: 7.

SEQ ID NO: 7:
PLKCFSRTFEDLTCFWDEE

In some embodiments, the polypeptide of the invention can act intracellularly (“cis” action) or extracellularly (“trans” action).

In some embodiments, the polypeptide of the invention is soluble. Advantageously, the solubility of the polypeptide of the invention allows that it can act extracellularly (“trans” action).

In some embodiments, the amino acid sequence of the polypeptide of the invention comprises an asparagine residue (Asn) at position 117, wherein said position is defined with respect to the amino acid sequence SEQ ID NO: 1.

In some embodiments, one or more amino acid(s) of the polypeptide of the invention are modified. Examples of post-translational modifications include, but are not limited to, phosphorylation, glycosylation, ubiquitination, nitrosylation, methylation, acetylation, lipidation and proteolysis. In one embodiment, one or more amino acid(s) of the polypeptide of the invention are glycosylated. As used herein, the term “glycosylated” defines the state of an amino acid with a glycan, i.e., polysaccharide, reversibly attached to one of its moieties. In a particular embodiment, the residue Asn at position 117 as described above is glycosylated.

In one embodiment, the glycosylation of Asn at position 117 is immature. In one embodiment, the immature glycan attached to Asn at position 117 is HexNAc(2)Hex(10), preferably HexNAc(2)Man(9)Glc(1). In another embodiment, the immature glycan attached to Asn at position 117 is HexNAc(2)Hex(9), preferably HexNAc(2)Man(9) or HexNAc(2)Man(8)Glc(1). In another embodiment, the immature glycan attached to Asn at position 117 is HexNAc(2)Hex(8), preferably HexNAc(2)Man(8).

In another embodiment, the glycosylation of Asn at position 117 is mature. In one embodiment, the mature glycan attached to Asn at position 117 is HexNAc(2)Hex(3) Fuc(1)Sias(1). In another embodiment, the mature glycan attached to Asn at position 117 is Hex(5)HexNAc(2)dHex(1).

In some embodiments, the amino acid sequence of the polypeptide of the invention comprises an asparagine residue (Asn) at position 117 which is unmodified.

In some embodiments, the amino acid sequence of the polypeptide of the invention comprises an asparagine residue (Asn) at position 117 which is not glycosylated. Without wanting to be bound to a theory, the Inventors showed that the glycosylation of the asparagine residue (Asn) at position 117 (N117) (wherein said position is defined with respect to the amino acid sequence SEQ ID NO: 1) of the polypeptide of the invention is not required for its “trans” action (i.e., extracellular action), i.e. is not required for its interaction with CALR mutant. Chachoua et al., 2016 discloses that the D1D2 fragment region of TPOR, corresponding to 258 amino acids, and its associated N-glycosylation site at N117 are required for CALR mutant activity. In the present invention, the Inventors define a fragment of TPOR as the fragment binding CALR mutant and being able to compete with activation of TPOR. Importantly, this identified fragment binds to TPOR even when N117 is lacking immature sugars. As a consequence, the polypeptide of the invention, even lacking glycosylation on N117, may compete with endogenous TPOR for its binding to CALR mutant. Therefore, the polypeptide of the invention advantageously inhibits TPOR activation by CALR mutant even when N117 is not glycosylated, can be soluble and can act from the extracellular space.

In some embodiments, the polypeptide of the invention binds to mutants of calreticulin (CALR) having a positively charged amino acid sequence in the C-terminus tail.

In some embodiments, the amino acid sequence of the polypeptide of the invention comprises at least one mutation compared to the amino acid sequence of SEQ ID NO: 2, wherein said mutation increases the affinity of the polypeptide for mutants of calreticulin.

As used herein, the term “mutation” refers to insertion(s), deletion(s), truncation(s) or substitution(s) of at least one amino acid by at least one natural and/or non-natural different amino acid. Non limitative examples of substitutions of amino acid with different chemical properties include the substitution of a positively charged arginine with a negatively charged glutamic acid, or the substitution of a polar asparagine with a non-polar tryptophan.

As used herein, the term “affinity” refers to the “binding affinity” between two molecules, herein preferably proteins or polypeptides. The binding affinity is typically assessed by determining the dissociation constant (Kd) or, for example, measuring the kinetics of a reaction (Michaelis constant, Km) using methods known in the art. In particular, when measuring the effects of an inhibitor, e.g. a competitive inhibitor or a non-competitive inhibitor, the inhibition constant (Ki) can be calculated.

In some embodiments, the polypeptide of the invention comprises at least one mutation which prevents or reduces the binding of the polypeptide to the thrombopoietin (THPO).

In some embodiments, the at least one mutation is localized on at least one domain of the polypeptide involved in the interaction with THPO. In some embodiments, the at least one mutation alters the tridimensional structure of the polypeptide, wherein the alteration of the tridimensional structure prevents the interaction with THPO.

In some embodiments, the polypeptide of the invention has a better affinity for CALR mutant than for THPO.

In one embodiment, a “better affinity” means a difference in affinity of at least 1.5-fold, 2-fold, 3-fold, 4-fold, 5-fold, 10-fold, or more. Comparison in affinity may be assessed by comparison Kd, Km, or Ki.

In some embodiments, the polypeptide of the invention, in particular the fragment region D1, has a better interaction with CALR mutant than the fragment region D1D2D3D4. Therefore, the polypeptide of the invention is more efficient to compete against CALR mutant than the fragment region D1D2D3D4.

In a one embodiment, the polypeptide is for use as a medicament.

The invention further relates to a fusion protein comprising a polypeptide as described hereinabove and at least one other polypeptide. As used herein in the context of the fusion protein, the polypeptide of the invention is referred to as “the first polypeptide” and the at least one other polypeptide is referred to as “the second polypeptide”.

In some embodiments, the second polypeptide increases stability of the first polypeptide. By increasing stability, it is meant the improvement of the resistance of the polypeptide to modifications or degradations, including but not limited to lysis, truncation, irreversible post-translational modifications or unfolding. In practice, the increase of stability results in an increase of the half-life of the polypeptide within the organism.

Methods for measuring protein or peptide stability are known in the art and include, without limitation, differential scanning calorimetry (DSC), bleach-chase, cycloheximide-chase, circular dichroism (CD) spectroscopy, SDS-PAGE electrophoresis.

In some embodiments, the second polypeptide decreases immunogenicity of the first polypeptide. By immunogenicity, it is meant the potency to trigger a detectable immune response within an organism, e.g., the production of antibodies, B cells, helper T cells, and/or cytotoxic T cells, directed specifically to one or more antigen.

Methods for measuring a T cell immune response are well known by the skilled artisan and include, without limitation, monitoring the production of IFN-gamma.

In one embodiment, the second polypeptide of the fusion protein is a Fc region of an immunoglobulin or a functional equivalent thereof. By Fc fragment of an immunoglobulin, it is meant the carboxy-terminal portions of both H chains held together by disulfides.

In some embodiments, the second polypeptide of the fusion protein is a Fc region of an immunoglobulin selected from the group comprising or consisting of IgA, IgD, IgE, IgG, IgM, and functional equivalents thereof.

In a preferred embodiment, the immunoglobulin is IgG, preferably IgG1 or IgG2. In another embodiment, the immunoglobulin is IgA. In another embodiment, the immunoglobulin is IgD. In another embodiment, the immunoglobulin is IgE. In another embodiment, the immunoglobulin is IgM.

In a preferred embodiment, the immunoglobulin is a human immunoglobulin. In another embodiment, the immunoglobulin is a non-human immunoglobulin.

In some embodiments, the immunoglobulin is modified, e.g., genetically and/or post-translationally modified. In practice, this modification is for increasing the stability of the fusion protein and/or its immunogenicity.

In one embodiment, the polypeptides of the fusion protein of the invention are disposed in a single, contiguous polypeptide chain.

In one embodiment, the fusion protein of the invention further comprises at least one peptide linker. In one embodiment, the polypeptides of the fusion protein of the invention are linked to each other through one or more peptide linkers.

Methods which are well known to those skilled in the art can be used to construct expression vectors containing the coding sequence of a fusion protein (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the fusion protein (fragment) (i.e., the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the fusion protein (fragment) of the invention, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (such as, e.g., Rous sarcoma virus). Other transcription control regions include those derived from vertebrate genes such as actin, heat shock protein, bovine growth hormone and rabbit α-globin, as well as other sequences capable of controlling gene expression in eukaryotic cells. Additional suitable transcription control regions include tissue-specific promoters and enhancers as well as inducible promoters (e.g., promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (TTRs).

Fusion proteins prepared as described herein may be purified by art-known techniques such as high-performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the fusion protein binds. The purity of the fusion protein can be determined by any of a variety of well-known analytical methods including gel electrophoresis, high pressure liquid chromatography, and the like.

In one embodiment, the fusion protein as described hereinabove is for use as a medicament.

The invention also relates to a nucleic acid comprising a sequence encoding the polypeptide or the fusion protein as described hereinabove.

In one embodiment, the nucleic acid encoding the fusion protein of the invention may be expressed as a single nucleic acid molecule that encodes the entire fusion protein or as multiple (e.g., two or more) nucleic acid molecules that are co-expressed. Polypeptides encoded by nucleic acid molecules that are co-expressed may associate through, e.g., disulfide bonds or other means, to form a functional fusion protein.

In one embodiment, the nucleic acid molecule is DNA. In another embodiment, the nucleic acid molecule is RNA, for example, in the form of messenger RNA (mRNA).

In one embodiment, the nucleic acid is linear. In another embodiment, the nucleic acid is circular.

In one embodiment, the nucleic acid as described hereinabove is for use as a medicament.

Another object of the invention is a vector comprising a nucleic acid according the invention. In some embodiments, at least one nucleic acid molecule is comprised in the one vector.

Within the scope of the instant invention, the expression “at least one nucleic acid” is intended to include 1, 2, 3, 4, 5, 6, 7, 8, 9, 10, 15, 20, 25, 30, 35, 40, 45, 50 or more nucleic acid molecules.

In some embodiments, the vector allows the controlled expression of at least one polypeptide and/or fusion protein. As used herein, the expression “controlled expression” is intended to refer to an expression that is controlled in time and/or space. In other words, the controlled expression of the polypeptide according to the invention may occur in a specific location of the body, such as, e.g., a specific organ, and/or for a specific time period.

In certain embodiments, the vector is a viral vector. Non limitative examples of viral vectors include adenovirus, adeno-associated virus (AAV), alphavirus, herpesvirus, lentivirus, non-integrative lentivirus, retrovirus, vaccinia virus and baculovirus.

In certain embodiments, the vector is a non-viral vector. Non limitative examples of non-viral vectors include inorganic particles (e.g., gold, calcium phosphate), lipidic emulsions, lipidic nanoparticles (e.g., liposomes), DNA-binding protein or peptide.

In one embodiment, the vector as described hereinabove is for use as a medicament.

The invention further relates to a pharmaceutical composition comprising a polypeptide, a fusion protein, a nucleic acid or a vector according to the invention, and a pharmaceutically acceptable vehicle.

In some embodiments, the pharmaceutically acceptable vehicle is selected in a group comprising or consisting of a solvent, a diluent, a carrier, an excipient, a dispersion medium, a coating, an antibacterial agent, an antifungal agent, an isotonic agent, an absorption delaying agent and any combinations thereof. The carrier, diluent, solvent or excipient must be “acceptable” in the sense of being compatible with the polypeptide, or derivative thereof, and not be deleterious upon being administered to an individual. Typically, the vehicle does not produce an adverse, allergic or other untoward reaction when administered to an individual, preferably a human individual.

For the particular purpose of human administration, the pharmaceutical compositions should meet sterility, pyrogenicity, general safety and purity standards as required by regulatory offices, such as, for example, the Food and Drugs Administration (FDA) Office or the European Medicines Agency (EMA).

In some embodiments, the carrier may be water or saline (e.g., physiological saline), which will be sterile and pyrogen free. Suitable excipients include mannitol, dextrose, lactose, starch, magnesium stearate, sodium saccharine, cellulose, magnesium carbonate, and the like.

Acceptable carriers, solvents, diluents and excipients for therapeutic use are well known in the pharmaceutical art, and are described, for example, in Remington's Pharmaceutical Sciences, Mack Publishing Co. (A. R. Gennaro ed. 1985). The choice of a suitable pharmaceutical carrier, solvent, excipient or diluent can be made with regard to the intended route of administration and standard pharmaceutical practice. The pharmaceutical compositions may comprise as, or in addition to, the carrier, excipient, solvent or diluent any suitable binder, lubricant, suspending agent, coating agent, or solubilizing agent. Preservatives, stabilizers, dyes and even flavoring agents may be provided in the pharmaceutical composition.

In some embodiments, the polypeptide, the fusion protein, the nucleic acid or the vector may be comprised in a delivery particle, in particular, in combination with other natural or synthetic compounds, such as, e.g., lipids, protein, peptides, or polymers.

Within the scope of the invention said delivery particle is intended to provide, or “deliver”, the target cells, tissue or organ with the polypeptide, nucleic acid or nucleic acid vector according to the invention.

In some embodiment, the delivery particle may be in the form of a lipoplex, comprising cationic lipids; a lipid nano-emulsion; a solid lipid nanoparticle; a peptide-based particle; a polymer-based particle, in particular comprising natural and/or synthetic polymers; and a mixture thereof.

In some embodiment, a polymer-based particle may comprise a synthetic polymer, in particular, a polyethylene imine (PEI), a dendrimer, a poly (DL-Lactide) (PLA), a poly(DL-Lactide-co-glycoside) (PLGA), a polymethacrylate and a polyphosphoesters.

In some embodiment, the delivery particle further comprises at its surface one or more ligand(s) suitable for addressing the polypeptide, the nucleic acid or the nucleic acid vector to a target cell, tissue or organ.

In one embodiment, the medicament is for the treatment and/or prevention of a blood cancer in a subject in need thereof. In a preferred embodiment, the medicament is for the treatment and/or prevention of myeloproliferative neoplasms (MPNs) in a subject in need thereof.

As used herein, the term “subject” refers to an animal, preferably a mammal, more preferably a human. In one embodiment, the subject is a man. In another embodiment, the subject is a woman. In one embodiment, the subject is a “patient”, i.e. the subject is awaiting the receipt of, or is receiving medical care, or was/is/will be the object of a medical procedure or treatment aiming to cure or treat the blood cancer, preferably a MPN, and/or alleviate the symptoms of the blood cancer, preferably a MPN. In some embodiments, the subject is monitored for the development of a blood cancer, preferably a MPN. In one embodiment, the subject is given a preventive treatment for a blood cancer, preferably a MPN. In one embodiment, the subject is an adult (for example a subject above the age of 18). In another embodiment, the subject is a child (for example a subject below the age of 18).

The invention also relates to a polypeptide, fusion protein or pharmaceutical composition according to the invention, for use in the treatment and/or prevention of a blood cancer, preferably a myeloproliferative neoplasm (MPN).

As used herein, MPN refers to a blood cancer type caused by a pathological constitutive activation of the JAK/STAT pathway that affects primarily hematopoietic stem cells and induces an abnormal expansion of cells of the myeloid lineage.

In one embodiment, the MPN is essential thrombocythemia (ET) or primary myelofibrosis (PMF).

In one embodiment, the polypeptide according to the invention alleviates, diminishes and/or suppresses the JAK/STAT constitutive activation inducing the MPN.

MPNs are also frequently associated with mutations of CALR, which is a soluble protein, resident of the endoplasmic reticulum (ER), that plays a role in the ER's protein quality control system as a chaperone, binding to the glycans of nascent N-glycosylated proteins, retaining these glycoproteins within the ER until properly folded.

In short, frameshift mutations of CALR may result in (i) loss of its ER retention, (ii) abnormal activation of TPOR and (iii) the presence of CALR at the cell surface and circulating CALR in the blood. One consequence of these mutations is the formation of a basic amino acids-rich carboxyl terminal (C-ter) tail in CALR that does not comprise its ER retention signal sequence.

In one embodiment, the MPN is induced, at least partially, by one or more mutation(s) in the gene encoding calreticulin (CALR). In one embodiment, the amino acid sequence of CALR mutant comprises at least one mutation compared to SEQ ID NO: 8, wherein SEQ ID NO: 8 is the amino acid sequence of wild type human calreticulin (UniProt ID number P27797).

In one embodiment, the one or more mutation(s) consist of insertions and/or deletions in exon 9, resulting in the generation of a positively charged amino acid sequence in the C-terminus of CALR.

As used herein, positively charged amino acids, interchangeably referred to as basic amino acids, are amino acids harboring a positive charge on their side chain at neutral pH, which encompass arginine, lysine, histidine, and all positively-charged unnatural amino acids.

In some embodiments, the CALR mutant inducing the MPN comprises the sequence SEQ ID NO: 9.

In some embodiments, the one or more mutation(s) of CALR results in a C-ter tail having an amino acid sequence selected from the group comprising or consisting of SEQ ID NO: 10 to SEQ ID NO: 45.

In some embodiments, the one or more mutation(s) of CALR results in a C-ter tail having an amino acid sequence as set forth in SEQ ID NO: 10 or SEQ ID NO: 11.

In one embodiment, the one or more mutation(s) of CALR results in a C-ter tail having an amino acid sequence as set forth in SEQ ID NO: 10, corresponding to CALR mutant L367fs*46, also referred to as CALR del52. In another embodiment, the one or more mutation(s) of CALR results in a C-ter tail having an amino acid sequence as set forth in to SEQ ID NO: 11, corresponding to CALR mutant K385fs*47, also referred to as CALR ins5.

In some embodiments, the polypeptide or fusion protein according to the invention binds to a mutant of CALR as described hereinabove.

In some embodiments, the MPN is induced by (i) one or more CALR mutation(s) and (ii) one or more mutation(s) in another gene(s). In some embodiments, the one or more CALR mutation(s) amplify the effect of the one or more mutation(s) in other gene(s).

In one embodiment, the one or more CALR mutation(s) and the one or more mutation(s) in other gene(s) occurs in the same cell. In another embodiment, the one or more CALR mutation(s) and the one or more mutation(s) in other gene(s) occurs in separate cells or groups of cells.

In some embodiments, the MPN is induced by one or more CALR mutation(s) and one or more mutation(s) in the gene encoding TPOR. In some embodiments, the mutation of TPOR is selected from the group comprising or consisting of R102P, P106L, G509N, and K39N. In some embodiments, the mutation of TPOR is R102P.

In some embodiments the polypeptide, fusion protein or pharmaceutical composition according to the invention inhibits the proliferation of cells expressing CALR mutant or cells expressing both CALR mutant and TPOR mutant.

In some embodiments, the polypeptide, the fusion protein, the nucleic acid, the vector or the pharmaceutical composition of the invention is administered in combination with a further anticancer agent or with an anticancer vaccine.

In certain embodiments, the further anticancer agent or vaccine is to be administered in combination with, concomitantly or sequentially, the polypeptide, the fusion protein, the nucleic acid, the vector or the pharmaceutical composition according to the invention.

In one embodiment, the further anticancer agent or vaccine is administered at the same time as the polypeptide, the fusion protein, the nucleic acid, the vector or the pharmaceutical composition according to the invention of the invention (i.e. simultaneous administration optionally in a co-formulation). In one embodiment, the further anticancer agent or vaccine is administered at a different time than the polypeptide, the fusion protein, the nucleic acid, the vector or the pharmaceutical composition according to the invention (i.e. sequential administration, where the further anticancer agent or vaccine is administered before or after the polypeptide is administered). In some embodiments, the further anticancer agent or vaccine may be administered in the same way as the polypeptide, the fusion protein, the nucleic acid, the vector or the pharmaceutical composition according to the invention, or by using the usual administrative routes for that further anticancer agent or vaccine.

In some embodiments, the polypeptide, nucleic acid, vector or pharmaceutical composition according to the present invention is to be administered orally, parenterally, topically, by inhalation spray, rectally, nasally, buccally, vaginally or via an implanted reservoir. The term administration used herein includes subcutaneous, intravenous, intramuscular, intraocular, intra-articular, intra-synovial, intrasternal, intrathecal, intrahepatic, intralesional and intracranial injection or infusion techniques.

In a preferred embodiment, the polypeptide, nucleic acid, vector or pharmaceutical composition of the present invention is to be administered parenterally, subcutaneously, intravenously, or via an implanted reservoir.

In some embodiments, the polypeptide, nucleic acid, vector or pharmaceutical composition of the invention is in a form adapted for injection, such as, for example, for intraocular, intramuscular, subcutaneous, intradermal, transdermal or intravenous injection or infusion.

Examples of forms adapted for injection include, but are not limited to, solutions, such as, for example, sterile aqueous solutions, dispersions, emulsions, suspensions, solid forms suitable for using to prepare solutions or suspensions upon the addition of a liquid prior to use, such as, for example, powder, liposomal forms and the like.

The treatment may consist of a single dose or a plurality of doses over a period of time. The polypeptide, nucleic acid, vector or pharmaceutical composition according to the invention may be formulated in a sustained release formulation so as to provide sustained release over a prolonged period of time such as over at least 2 or 4 or 6 or 8 weeks. Preferably, the sustained release is provided over at least 4 weeks.

In certain embodiments, the effective amount of the polypeptide, nucleic acid, vector or pharmaceutical composition to be administered may depend upon a variety of parameters, including the material selected for administration, whether the administration is in single or multiple doses, and the subject's parameters including age, physical conditions, size, weight, gender, and the severity of the cancer to be treated.

In some embodiments, the polypeptide, nucleic acid, vector or pharmaceutical composition according to the invention is administered to the subject in need thereof in a therapeutically effective amount.

By “therapeutically effective amount”, it is meant a level or amount that is necessary and sufficient for slowing down or stopping the progression, aggravation, or deterioration of one or more symptoms of cancer; or alleviating the symptoms of cancer; or curing cancer, without causing significant negative or adverse side effects to the individual. In certain embodiments, an effective amount of the polypeptide according to the invention may range from about 0.001 mg to about 3,000 mg, per dosage unit, preferably from about 0.05 mg to about 1,000 mg, per dosage unit.

Within the scope of the instant invention, from about 0.001 mg to about 3,000 mg includes, from about 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg and 3,000 mg per dosage unit.

In certain embodiments, the polypeptide according to the invention is to be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day.

In some embodiments, an effective amount of the nucleic acid or vector to be administered may range from about 1×10⁵ to about 1×10¹⁵ copies per dosage unit.

Within the scope of the instant invention, from about 1×10⁵ to about 1×10¹⁵ copies includes 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴ and 1×10¹⁵ copies, per dosage unit.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets. The formulations for use in the present invention may further include other agents conventional in the art having regard to the type of formulation in question, for example those suitable for oral administration may include flavoring agents.

Another object of the present invention is a kit (i) at least one polypeptide, at least one fusion protein, at least one nucleic acid, at least one vector, at least one pharmaceutical composition or at least one vaccine according to the invention, and (ii) means to administer said polypeptide, fusion protein, nucleic acid, vector or pharmaceutical composition. In one embodiment, the kit is for treating and/or preventing a disease, preferably a blood cancer, more preferably a MPN.

In certain embodiments, the means to administer the polypeptide, the fusion protein, the nucleic acid, the vector or the pharmaceutical composition according to the invention may include a syringe, a trocar, a catheter, a cup, a spatula, and the likes.

In one embodiment, the kit further comprises an anticancer agent or vaccine, preferably for treating a blood cancer, more preferably for treating a MPN.

In some further aspects, the invention also relates to the use of the polypeptide, the fusion protein, the nucleic acid, the vector or the pharmaceutical composition according to the invention for the manufacture or the preparation of a medicament.

The invention also relates to a polypeptide, fusion protein, nucleic acid, vector or pharmaceutical composition according to the invention, for the manufacture of a medicament for treating and/or preventing a blood cancer, preferably a myeloproliferative neoplasm (MPN), preferably a MPN induced by one or more mutation(s) in the gene encoding calreticulin (CALR).

The invention also relates to a method for treating and/or preventing a blood cancer, preferably a myeloproliferative neoplasm (MPN), preferably a MPN induced by one or more mutations in the gene encoding calreticulin (CALR), in a subject in need thereof comprising administering to said subject a therapeutically effective amount of the polypeptide, fusion protein, nucleic acid, vector or pharmaceutical composition according to the invention.

The present invention also relates to a polypeptide, fusion protein, nucleic acid, vector or pharmaceutical composition according to the invention, for use in inhibiting the expansion of cancer cells.

The present invention also relates to a method for inhibiting the expansion of cancer cells in an individual in need thereof, comprising at least the step of administering to the individual a therapeutically effective amount of polypeptide, fusion protein, nucleic acid, vector or pharmaceutical composition according to the invention.

The present invention also concerns a polypeptide, fusion protein, nucleic acid, vector or pharmaceutical composition according to the invention, for use in improving the overall survival of an individual having cancer.

The present invention also concerns a method for improving the overall survival of an individual having cancer, comprising at least the step of administering to the individual a therapeutically effective amount of polypeptide, fusion protein, nucleic acid, vector or pharmaceutical composition according to the invention.

The present invention also concerns a polypeptide, fusion protein, nucleic acid, vector or pharmaceutical composition according to the invention, for use in improving the prognosis of an individual having cancer.

The present invention also concerns a method for improving the prognostic of an individual having cancer, comprising at least the step of administering to the individual a therapeutically effective amount of polypeptide, fusion protein, nucleic acid, vector or pharmaceutical composition according to the invention.

### BRIEF DESCRIPTION OF THE DRAWINGS

FIGS. 1A to 1I shows that CALR del52 mutant tail directly interacts with TpoR extracellular domain. FIG. 1A: Proliferation assay. BaF3 cells stably expressing hTpoR in pMX-IRES-GFP were infected with indicated CALR variants or an empty vector (pMSCV-IRES-mCherry) and sorted by FACS. 250,000 were washed and seeded in 10 ml of complete culture medium without cytokine and counted each day using an automated cell counter (n=3). Values represent average of 3 independent experiments (±SD) with technical triplicates (n=6-9). FIG. 1B: NanoBRET between NanoLuc-TpoR and CALR del52-HaloTag full length or truncated from the N-terminus. Shown are average 5 independent experiments (±SD) (N=5, n=20). Two-ways ANOVA followed by SIDAK multiple comparison test. ****: p<0.0001, ns: non-significant. FIG. 1C-1E: Theoretical profile of specific versus non-specific interaction in a Donor Saturation Assay (DSA) experiment. HEK293T were transfected with increasing amount of HaloTag constructs and fixed amount of NanoLuc donor. Negative control consists in an empty NanoLuc vector (n=4). FIG. 1C: DSA of CALR del52 full length. FIG. 1D: DSA of CALR del52 P-C domain. FIG. 1E: DSA of CALR del52 C-domain. FIG. 1F: Representative co-immunoprecipitation of HA-hTpoR with CALR del52-FLAG full length or N-terminal truncations as indicated. FIG. 1G: ELISA of CALR del52 species from HEK293T cell culture supernatants. Values represent average of 3 ELISA experiments. One-way ANOVA followed by Tukey multiple comparison test. ****: p<0.0001. FIG. 1H: Surface localization of CALR del52 full lenth or P-C domain in presence of hTpoR or an empty vector. Flow cytometry analysis using an anti-FLAG antibody and a secondary anti-mouse antibody coupled to APC on HEK293T co-transfected with the indicated constructs. FIG. 1I: STAT5 transcriptional activity with hTpoR and indicated CALR truncations. HEK293T were transiently transfected with HA-hTpoR and CALR truncations along with cDNAs coding for STAT5, JAK2 and SpiLuc Firefly luciferase reporter reflecting STAT5 transcriptional activity and normalized with a control reporter (pRLTK) containing Renilla luciferase. Shown are average 3 independent experiments (±SD) performed in triplicates (N=3, n=9). Two-ways ANOVA followed by SIDAK multiple comparison test. ***: p<0.001, ****: p<0.0001.

FIGS. 2A to 2F concern CALR del52-TpoR interaction and mutant tail driven dimerisation. FIG. 2A-C: Deuterium uptake of representative peptides from CALR del52 alone or in complex with TpoR ECD corresponding to the C-terminus of CALR del52 at 5 different exchange time points. The peptide shown in FIG. 2A corresponds to the end of the C-domain common to CALR WT and CALR del52 which does not show differential uptake. The peptide shown in FIG. 2B contains the mutant tail and show high differential uptake. The peptide shown in FIG. 2C corresponds to the last amino acids of the mutant tail. FIG. 2D: Native western blot of indicated CALR mutants, with or without reducing agent (DTT). Staining with Coomassie Blue. FIG. 2E: Co-immunoprecipitation of CALR del52-HA full length by CALR del52-FLAG full length or truncated to assess dimerization. Shown are representative western blots in denaturing conditions. FIG. 2F: Crosslinking study of hTpoR dimerisation in presence of Tpo, CALR del52 full length of C-terminal truncations. Shown is a representative western blot in denaturing and reducing conditions showing hTpoR monomers and o-PDM crosslinked dimers in the indicated conditions.

FIGS. 3A to 3G shows interaction of TpoR D1D2 with CALR del52. FIG. 3A: Representative co-immunoprecipitation of HA-hTpoR ECD domains with CALR del52-FLAG or CALR del52 Y109F/D135L-FLAG as indicated. FIG. 3B: Quantification of relative co-immunoprecipitation of TpoR species by CALR del52 (mutated or not). Western blot quantification performed with ImageJ. Shown are the ratios (+SD) of TpoR species on CALR del52 normalized for TpoR species expression in whole lysates (N=2, n=6). Two-ways ANOVA followed by SIDAK multiple comparison test. ***: p<0.001, *:p<0.05 ns: non-significant. FIG. 3C: NanoBRET between NanoLuc-TpoR subdomains and CALR del52-HaloTag full length or truncated from the N-terminus. Shown are average 2 independent experiments (±SD) (N=3, n=12). FIG. 3D-3F: Deuterium uptake of representative peptides from CALR del52 alone or CALR del52-D1D2 complex corresponding to key regions described in the text. The peptide shown in FIG. 3D corresponds to the amino acids 50-74 of the N-domain. The peptide shown in FIG. 3E contains residues C105 and Y109 involved in glycan binding. The peptide shown in FIG. 3F contains the mutant C-terminus. FIG. 3G: Relative differential deuterium uptake between CALR del52 in complex with sD1D2 containing immature glycans and CALR del52 alone at 60 min incubation in deuterium. Dots represent individual peptides detected by mass spectrometry. The Y axis represents the relative deuterium exchange differential. Positive values denote regions of CALR del52 that are more protected in presence of sD1D2 and negative values represent regions that are less protected in presence of sD1D2.

FIGS. 4A to 4C concern interaction sites between CALR del52 mutant tail and TpoR D1D2. FIG. 4A: Relative deuterium uptake analysis between TpoR full ECD with mature glycans in absence or presence of CALR del52 at 1 hour incubation in deuterium. Dots indicated as protected show significant differential deuterium intake (p<0.001) with the peptide-level significance testing (hybrid mode) as described (Lau et al., 2021). FIG. 4B: Deuterium uptake of the FSRTFED peptide from mature TpoR full ECD (D1D4) alone or in complex with CALR del52 (CALR del52_D1D4) at 5 different exchange time points. FIG. 4C: STAT5 transcriptional activity with hTpoR and indicated CALR truncations. HEK293T were transiently transfected with HA-hTpoR and CALR truncations along with cDNAs coding for STAT5, JAK2 and SpiLuc Firefly luciferase reporter reflecting STAT5 transcriptional activity and normalized with a control reporter (pRLTK) containing Renilla luciferase. Shown are average 3 independent experiments (±SD) performed in triplicates (N=3, n=9). Two-ways ANOVA followed by SIDAK multiple comparison test. ***: p<0.001, ****: p<0.0001.

FIGS. 5A to 5D are set of graphs supporting the principle of the inhibition of CALR del52 binding to the fusion protein comprising TPOR domains (TPOR-Fc) in Myeloproliferative neoplasms. Competition experiments with increasing concentrations of soluble D1D2D3D4-Fc (FIG. 5A), D1D2-Fc (FIG. 5B) and D1-Fc (FIG. 5C). Experiments performed in cell co-expressing equivalent cDNA of TpoR/TPOR coupled to NanoLuciferase and CALR del52 coupled to HaloTag. The interaction between TPOR and CALR del52 is measured based on the BRET ratio. FIG. 5D illustrates NanoBRET signal used as a surrogate for binding between TpoR full length and CALR del52 in presence of increasing amount of human D1 protein with the N117Q mutation.

FIG. 6 is an histogram showing inhibition of CALR mutant-TPOR interaction in trans on living cells. Detection of CALR del52 at the cell surface of cells after incubation with indicated Fc-fusion protein or controls is shown.

FIG. 7 is a graph showing the in vivo efficacy of soluble D1 as inhibitor of CALR mutant. The graph represents blood platelets concentrations of indicated mice from 4 to 24 weeks.

FIG. 8 is a graph illustrating NanoBRET signal used as a surrogate for binding between TPOR full length and CALR del52 in presence of increasing amount of human D1 WT and mutants in the FFPLHLWLV motif.

FIG. 9 is a model of the tridimentionnal structure representing the interaction of TPOR and CALR del52 mutant during the formation of a tetramer complex.

FIG. 10 illustrates NanoBRET signal used as a surrogate for binding between TPOR full length and CALR del52 for binding between TPOR full length and CALR del52 in presence of increasing amount of human D1 WT and mutants in motifs QDV, SDSE, FSR, WDEE or EAAP.

### EXAMPLES

The present invention is further illustrated by the following examples.

As used herein, the term “mutant tail” refers to the C-terminal tail of CALR del52 having the amino acid sequence of SEQ ID NO: 50.

### Example 1: CALR Del52 Mutant Tail Directly Interacts with TpoR Extracellular Domain and Mediates TpoR and CALR Dimerization

### Materials and Methods

Mutagenesis: All CALR del52 mutants were made alternatively using the QuickChange (Agilent), the KLD enzyme mix (NEB) procedure following the manufacturer instruction or purchased from Genscript. All constructs were verified by sequencing.

Ba/F3 proliferation assay: Ba/F3 stably were first transduced with human TpoR in pMX-IRES-GFP and CALR variants or an empty vector (pMSCV-IRES-mCherry) and sorted by FACS for similar levels of GFP and mCherry. 250,000 cells were washed and seeded in 10 ml RPMI, 10% FBS without cytokine and counted each day using a Coulter automated cell counter in triplicates. The experiments were performed in three different biological replicates (N=3).

BRET: cDNAs coding for TpoR and the erythropoietin receptor (EpoR) were cloned into a modified pNL-N vector (Promega) to generate an N-terminal fusion of the Nano-luciferase to the receptors. cDNAs coding for WT and the del52 CALR mutants were cloned into the pHT-C vector to generate HaloTag fused constructs (Promega). HEK-EBNA cells transiently transfected with those constructs were analyzed for bioluminescence resonance energy transfer (BRET) on a GloMax Discover multiplate reader (Promega) at 37° C. using the 450BP (donor) and 600LP (acceptor) built-in filters.

Co-immunoprecipitation and Western Blotting: HEK293T were plated in 10 cm dishes and transiently transfected with cDNA coding for the indicated constructs. Confluent cells were lysed 48 h post-transfection with NP-40 buffer. After pre-clearing, samples were incubated with anti-FLAG antibody (Genscript, Cat. No. A00187) at 2 μg/ml or corresponding isotype control (Genscript Cat. No. A01730) overnight at 4° C. Bound proteins were pulled down with 40 μL/ml of rProtein G Agarose (ThermoFisher, 20397) for 3 hours at 4° C. Samples were then centrifuged, washed three times and immunoprecipitated proteins were analyzed by SDS-PAGE followed by revelation with an anti-HA antibody (Roche, 3F10) for HA-hTpoR, HA-CALR del52 or anti-CALR mutant tail (SAT602).

ELISA: For measuring the level of soluble CALR mutant species from HEK293T supernatant 48 h post transfection, a polyclonal rabbit antibody (SAT602 provided by Myelopro, Vienna, Austria) was used for coating of the ELISA plates. This antibody was generated against a peptide derived from the CALR mutant C-terminal sequence. After blocking (5% BSA+0.05% Tween-20 in PBS), plates were probed with culture medium samples diluted in blocking buffer and purified mutant-CALR protein produced in Expi-293F cells (Thermo Fisher Scientific, Merelbeke, Belgium) was included as standard for quantification. For detection, an anti-CALR antibody (FMC75, Abcam, Cambridge, United Kingdom) in combination with an anti-mouse IgG-HRP antibody (Southern Biotech, Birmingham, AL) was used and TMB (3,3′, 5,5′-Tetramethylbenzidine, Thermo Fisher Scientific, Merelbeke, Belgium) was added as substrate. Absorbance was measured with a microplate reader (SpectraMax i3, Molecular Devices, Silicon Valley, CA) at 450 and 620 nm.

Flow cytometry: For assessment of CALR del52 species cell surface localization, HEK293T were transiently transfected with indicated CALR del52 species and full length human TpoR (hTpoR) or an empty vector 48 h prior to the experiment. Cells were detached without trypsination and stained with anti-FLAG antibody or IgG control (primary) and APC coupled Goat anti-mouse antibody (secondary). Cells transfected with single fluorescence vector (GFP or mCherry) or compensation beads stained with APC-coupled antibody were used for compensation controls. 30,000 events of cells co-transfected with the two constructs were acquired for each condition on BD LSRFortessa™ Cell Analyzer. Post-acquisition analysis was performed with FlowJo. For assessment of TpoR mutant cell surface localization, HEK293T were transfected with indicated mutant or an empty control vector. 48 h post-transfection, cells were processed as described above and stained with anti-HA coupled to APC antibody. 30,000 events of transfected cells were acquired for each condition on using a FACSVerse™ and the percentage of positive cells for HA-APC staining was determined amongst the transfected cells.

Hydrogen-Deuterium Exchange Mass Spectrometry (HDx-MS): Hydrogen-Deuterium Exchange Mass Spectrometry was performed with a Waters nanoAcquity UPLC with HDx technology coupled with Synapt G2-Si. All purified recombinant proteins were used at 20 μM concentration in equilibration buffer (5 mM K2HPO4, 5 mM KH2PO4 dissolved in H2O, pH 7). For interaction analysis between recombinant mature D1D2D3D4 and CALR del52, proteins were first mixed together at a 1:1 molarity for 30 minutes at room temperature followed by 3 hours at 4° C. Proteins were then kept at 0° C. Labelling was performed with a 20-fold dilution of samples in labelling buffer (5 mM K2HPO4, 5 mM KH2PO4 dissolved in D20, pD 7) for 6 different incubation times (0, 0.25, 1, 5, 20 and 60 minutes) at 20° C. After incubation, the reaction was quenched using a 1:1 dilution in the quench buffer (0.05 M K2HPO4, 0.05 M KH2PO4 with 30 mM TCEP, pH 2.3) prior to injection into a pepsin column (Enzymate BEH Pepsin 2.1×30 Column, Waters CAT. 186007233) with dynamic flowrate of 150-75 μL/min. All mixes were performed automatically by a PAL-RTC robot station. Peptides resulting from the pepsin digestion were captured on a ACQUITY BEH C18 1.7 μM VANGUARD Pre-column (Waters Cat. 186009375), separated on a ACQUITY UPLC C18 1.7 μM 1.0×100 mm column (Waters Cat. 186002346) and electrosprayed into the Waters SYNAPT G2-Si quadrupole time-of-flight mass spectrometer. Measurements were performed in HDMSe mode with ion mobility. Lock mass correction was performed with infusion of leucine-enkephalin (m/z=556.277). The peptides were identified from triplicates using the PLGS3.0 software (Waters). The peptides identified were further analyzed with DynamX 3.0 (Waters) using a tolerance of 10 ppm, a maximum length of 35 a.a., a minimum products per amino acid of 0.2 and requiring that each peptide was identified in 3 out of 3 replicates. All peptides were visually validated based on retention time, drift time and isotopic m/z. Data was statistically analyzed using Deuteros 2.0 with peptide-level significance testing (Lau et al., 2021).

### Results

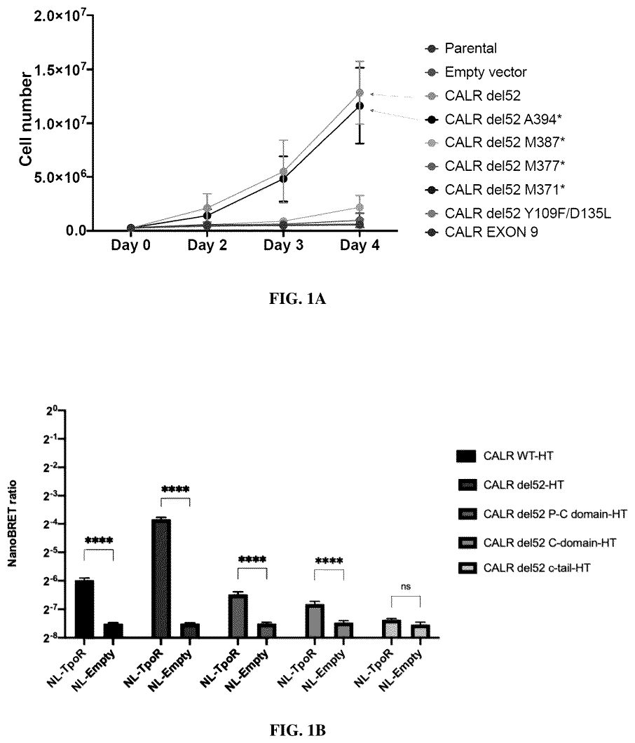

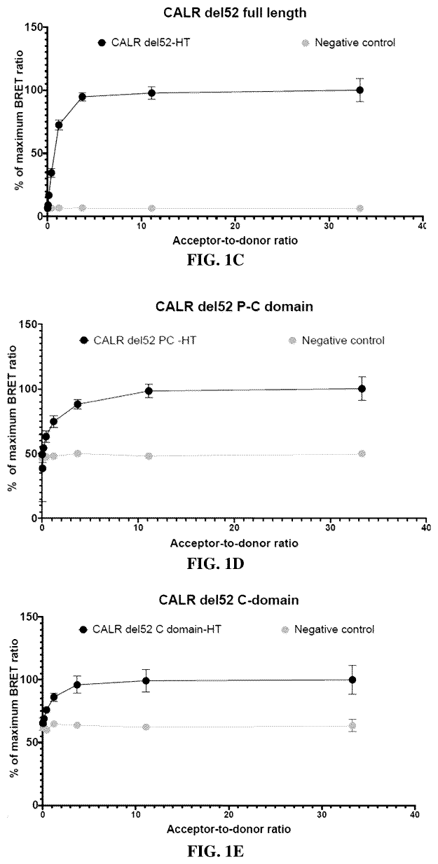

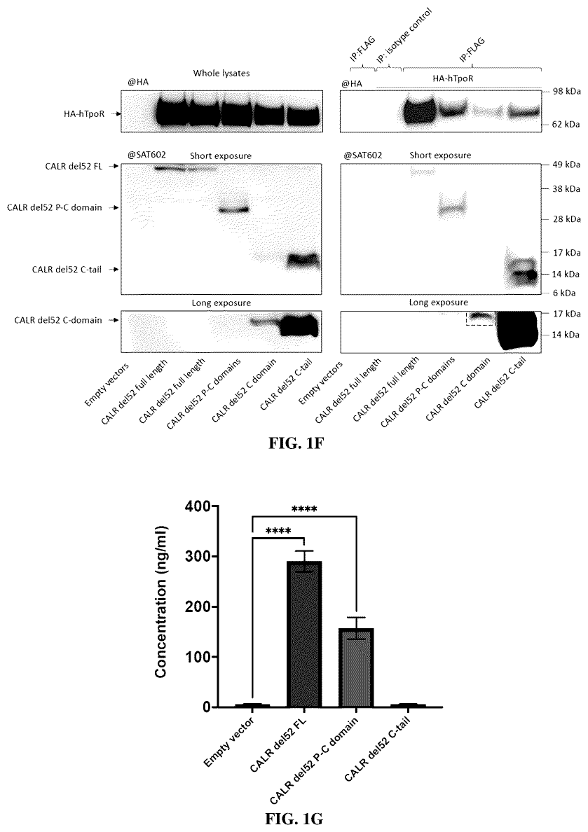

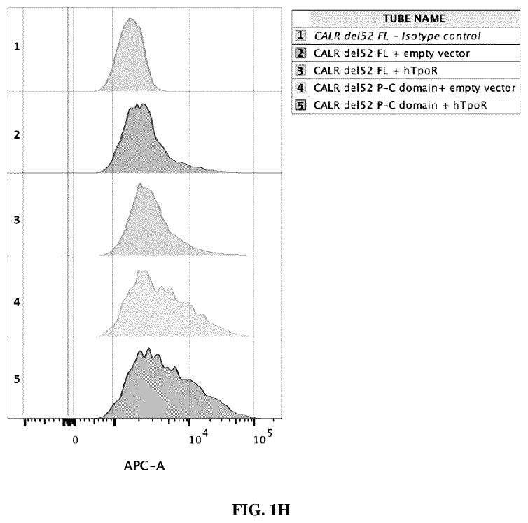

Calreticulin domains includes a globular N-domain (position 16-197), a central proline-rich P-domain (position 198-308) and an acidic C-domain (position 309-417). In some CALR mutants, in particular CALR del52, the C-terminus tail is positively charged. To investigate the role played by the positively charged mutant tail, progressive truncatures of the C-terminus of CALR del52, the prototype of type I CALR mutant, were tested. Truncations of the C-ter part until position 394, thus removing the last 18 amino acids, allowed autonomous growth of cytokine-dependent hematopoietic cell lines (Ba/F3) similar to that induced by full length CALR del52 (FIG. 1A, “CALR del52 A394*” compared to “CALR del52”). Further truncation up to position 387 still allowed low autonomous proliferation (FIG. 1A, “CALR del52 M387*”) while further truncations completely abolished the activity of the CALR (FIG. 1A, “CALR del52 M377*”, “CALR del52 M371*”, and “CALR EXON 9” which represents the deletion of exon 9).

Importantly, the CALR del52 Y109F/D135L mutant, which abolishes glycan-dependent binding and disturbs the helicity of the mutant tail, did not allow Ba/F3 autonomous proliferation (FIG. 1A, “CALR del52 Y109F/D135L”).

N-terminal truncations of CALR del52 were also created to probe whether the mutant tail of CALR del52 may also mediate direct binding to the receptor in absence of immature glycans. Bioluminescence Resonance Energy Transfer (BRET) was used to measure direct interaction between TpoR and N-terminal truncations of CALR del52 directly in living cells. Strikingly, complete deletion of the N-domain (CALR del52 P-C domain-HT) or both the N and P-domain (CALR del52 C-domain-HT) of CALR del52 did not completely block the direct interaction with the receptor (FIG. 1B).

The specificity of the interaction was validated by Donor Saturation Assay (DSA) where increasing amount of ligand (i.e. CALR variants) leads to rapid signal saturation due to occupation of all receiver binding sites (FIGS. 1C-E).

When only the mutant tail was present, a small, but not significant binding to TpoR, was observed, suggesting that the tail required at least part of the C-domain for proper folding and stability when fused to the HaloTag construct. To validate these results and probe binding of the C-tail only to TpoR, co-immunoprecipitation were performed with CALR del52 constructs containing a FLAG tag at the C-terminus. Interaction between TpoR and C-terminal truncations of CALR del52 up to a fragment corresponding exclusively to the mutant tail was detected (FIG. 1F).

Then, C-terminal fragments of CALR del52 were tested whether they behaved similarly as the full length in functional assays. ELISA on cell supernatant were used to measure secretion of CALR del52 P-C domain or mutant tail alone compared to full length CALR del52. Interestingly, the P-C domain was strongly secreted whereas the mutant tail alone was not (FIG. 1G).

Flow cytometry was then used to assess surface localization of CALR del52 full length or P-C domain in presence or absence of TpoR. Both CALR del52 full length and even more the P-C domain showed strong surface expression (FIG. 1H). Interestingly, both CALR del52 species (full length and P-C domain) had higher surface localization in cells co-expressing TpoR, suggesting that the latter retain them at the cell surface (FIG. 1H).

Finally, a functional transcriptional assay was used to show that the P domain fused to the C domain of CALR del52 is sufficient to induce small but significant STAT5 transcriptional activity via TpoR but not EpoR (FIG. 1I).

Together these results show that direct binding of CALR del52 to TpoR is mediated both by the mutant tail and N-domain interaction with immature sugars but that activation is strictly dependent on the presence of the mutant tail. The latter is further consolidated by the glycan-dependent interaction.

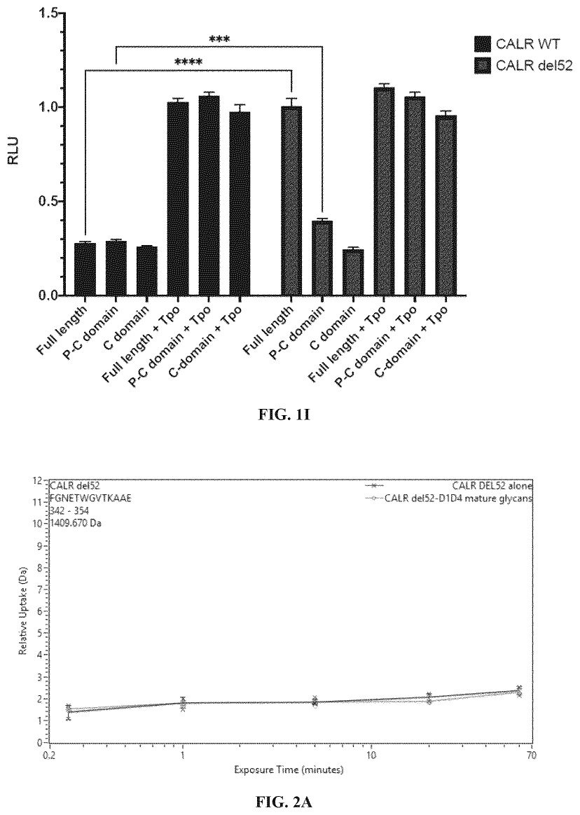

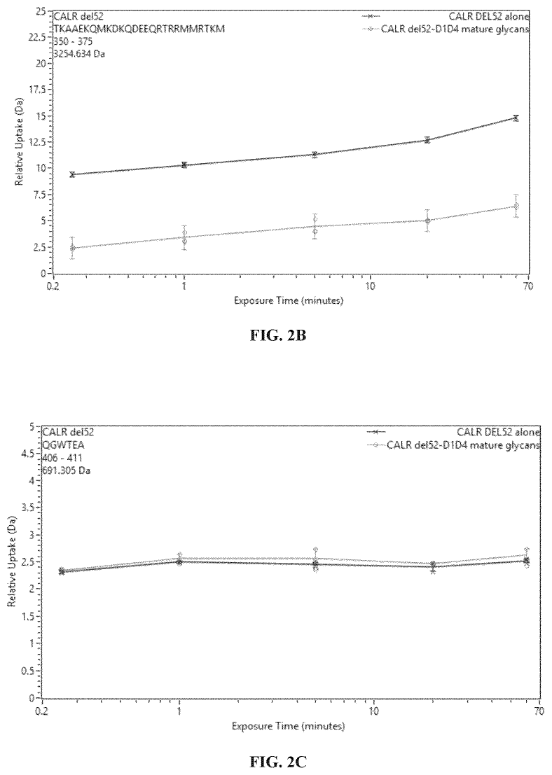

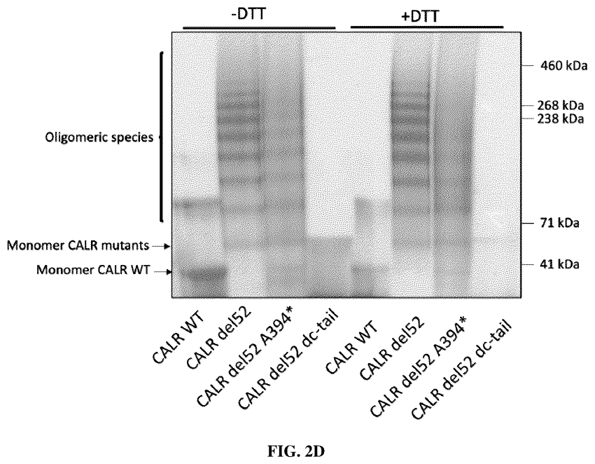

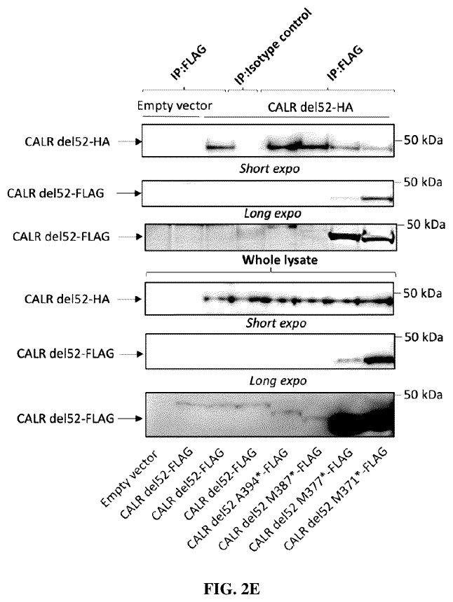

Subsequently, HDx-MS was used to map any possible interaction sites between 3 CALR del52 mutants (namely FGNETWGVTKAAE—SEQ ID NO: 51; TKAAEKQMKDKQDEEQRTRRMMRTKM—SEQ ID NO: 52; and QWGTEA—SEQ ID NO: 53) and the complete extracellular domain of the TpoR containing mature glycans. The two recombinant proteins were produced independently and mixed in solution at a 1:1 molar ratio prior to deuterium exchange. Results revealed significant (p<0.001) exchange differential in multiple peptides containing the mutant tail (FIG. 2A), confirming a direct interaction between the mutant tail and the mature TpoR extracellular domain. This differential exchange was not observed in the very last residues of the mutant tail encompassing residues 406QWGTEA411 (SEQ ID NO: 53, FIG. 2C), showing that the very last part of the mutant tail is not involved in interaction.

Because oligomerisation of CALR mutants is key for TpoR activation (Araki et al., 2019; Rivera et al., 2021), and that truncations before the C-terminal cysteines did not affect proliferation, the oligomeric profile of CALR del52 species were compared with and without reducing agent in native conditions. No difference in the oligomeric profile was observed in presence or not of a reducing agent (DTT) and both CALR del52 A394* and CALR del52 full length gave rise to similar oligomeric species (FIG. 2D). In contrast, oligomeric species disappeared completely with CALR Δc-tail, supporting the fact that the mutant tail is required for oligomerization, which is independent of the C-terminal cysteines (FIG. 2D).

Using the same set of deletions of CALR del52 as in the functional assay (see FIG. 1A), the role of the mutant tail in dimerization was further probed by co-immunoprecipitation of HA-tag full length CALR del52 with the same FLAG-tag truncations of CALR del52 as used in the proliferation assay. We observed strong co-immunoprecipitation of CALR del52 full length, CALR del52 A394* and CALR del52 M387* (FIG. 2E). This is in line with the conserved ability of these truncations to induce Ba/F3 independent proliferation. Despite their strongly increased stability, further truncations (CALR del52 M377* and CALR del52 M371*) led to sharp decrease in immunoprecipitation of HA-CALR del52 full length (FIG. 2E), confirming that the mutant tail is directly involved in CALR del52 dimerization.

A crosslinking approach was then used to study TpoR dimerization with CALR del52 full length or C-terminal truncations. To detect TpoR dimerization through cysteine crosslinking, the intracellular domain of the receptor after JAK2 binding sites was truncated to remove intracellular cysteines and inserted the L508C point mutation, homologous to murine TpoR L501C, putting the cysteine residue in a dimeric orientation that was showed to be active (Staerk et al., 2011). These TpoR truncations retain the ability to be activated. In line with the functional assays, truncations until M387 (CALR del52, CALR del52 A394* and CALR del52 M387*) still enabled dimerization of TpoR transmembrane domain while further truncations (CALR del52 M377* and CALR del52 M371*) did not allow the formation of dimers (FIG. 2F).

### Example 2: TpoR D1D2 is Sufficient to Mediate Full Binding to CALR Del52 Through Glycans and Mutant Tail

### Materials and Methods

For co-immunoprecipitation/Western Blotting, BRET, HDx-MS: see example 1.

### Results

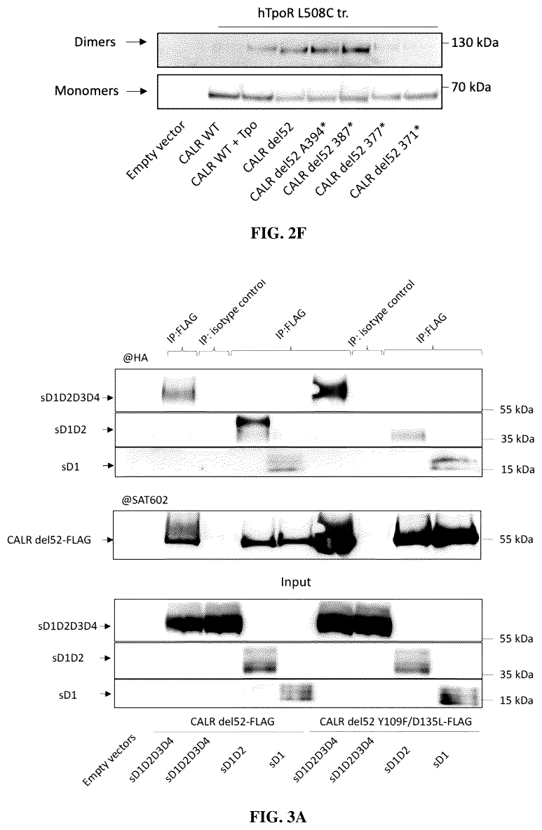

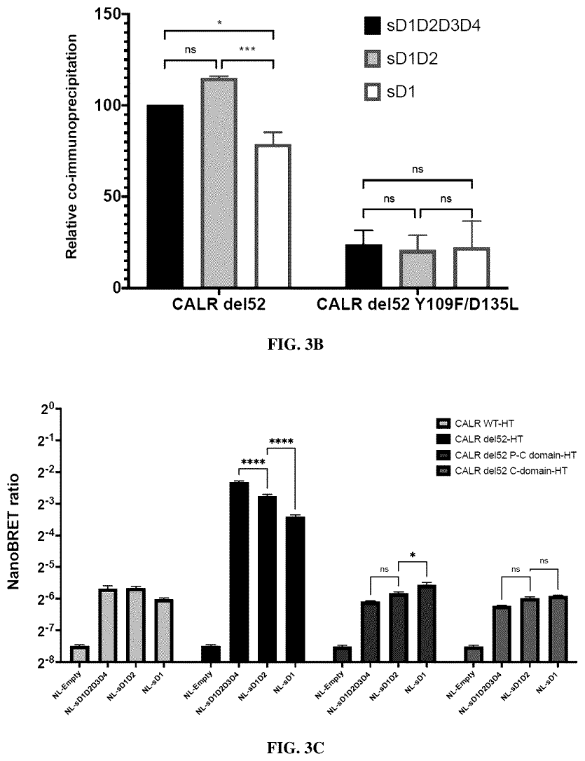

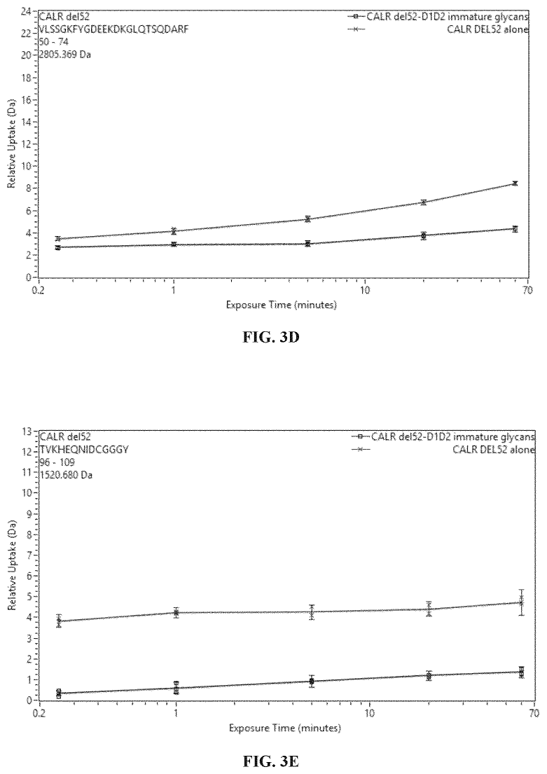

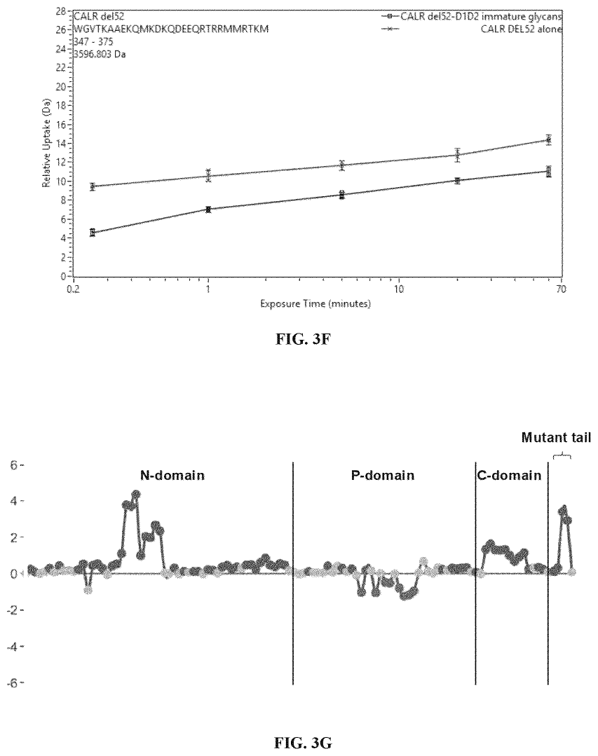

Previous findings showed that activation of the TpoR by CALR del52 was mediated by the extracellular domain of TpoR and was dependent on glycosylation of Asn117 and, to a lesser extent of Asn178, present on TpoR D1 and D2 domain, respectively (Chachoua et al., 2016; C. Pecquet et al., 2019). Using co-immunoprecipitation of C-terminal truncations of TpoR ECD, deletion of the D3D4 was shown to not impact binding of CALR del52 to the receptor (FIG. 3A-B with CALR del52-FLAG, sD1D2). Deletion of the D2 domain led to small but significant reduction of co-immunoprecipitation compared to the D1 domain alone (FIG. 3A-B with CALR del52-FLAG, sD1).

Next, the CALR del52 Y109F/D135L mutant which lacks ability to bind glycans (Kapoor et al., 2004) was used. As expected, loss of glycan-dependent binding led to a sharp decrease in co-immunoprecipitation ratios (FIG. 3A-B with CALR del52 Y109F/D135L-FLAG). Strikingly, no differences in co-immunoprecipitation ratio were detected between D1 alone and D1D2 in absence of glycan binding (FIG. 3A-B with CALR del52 Y109F/D135L-FLAG, sD1 compared to sD1D2), suggesting that CALR del52 mutant tail interacts exclusively with TpoR D1 domain.

BRET assays between CALR del52 truncations and subdomains of TpoR ECD were then performed to measure direct interaction in living cells. Confirming co-immunoprecipitation results, a reduction of BRET signal between D1D2 and D1 with full length CALR del52 was observed (FIG. 3C with CALR del52-HT, NL-sD1D2 compared to NL-sD1). In contrast, binding of CALR del52 P-C domain or C-domain only to the receptor ECD was similar or even slightly higher after truncation of D3D4 or D2D3D4 (FIG. 3C with CALR del52 P-C domain-HT and CALR del52 C-domain-HT, NL-sD1D2 and NL-sD1).

Together with results above, this confirmed that CALR del52 mutant tail directly interacts with the D1 domain of the receptor.

Subsequently, CALR del52-D1D2 produced as a complex in Schneider cells was purified. Briefly, the extracellular domain of the human TpoR and human CALR mutant del52 produced in Drosophila S2 cells (Thermo Fisher Scientific) were fractionated by using size-exclusion chromatography by loading on a Superdex 200 Increase 10/300 column (GE Healthcare). Elution was performed at 0.5 mL/min with buffer TNG (Tris-NaCl-glycerol) pH 7.5, and 0.5 mL fractions were collected and analyzed by sodium dodecyl sulfate-polyacrylamide gel electrophoresis and western blot. Fraction proteins were immunoprecipitated with anti-His5 antibody (Qiagen) and treated with Endoglycosidase-H (NEB) or N-glycosidase F (NEB). HDx-MS was used to detect all interaction sites between CALR del52 and TpoR D1D2 (sD1D2) produced in the same cells. Comparison of deuterium uptake between CALR del52 alone and the CALR del52-TpoR D1D2 complex showed that the CALR mutant interacted with TpoR via two major domains. First, the highest deuterium uptake differential was observed in the putative glycan binding site of calreticulin (FIG. 3E). This region included notably C105, Y109, D135 and W319, all reported to be key for binding of sugar moieties (Gopalakrishnapai et al., 2006). Importantly, this region was not involved in binding of mature TpoR as shown above, confirming that it is specifically involved in interaction with immature glycans. Second, multiple peptides containing CALR del52 mutant tail equally showed high degrees of differential uptake between the CALR del52-sD1D2 complex and CALR del52 alone (data not shown). This confirmed that the mutant tail is involved not only in binding to mature forms of the receptors, but also to immature TpoR, the interaction being further consolidated by strong interactions between the CALR glycan binding pocket and immature sugar moieties on Asn117 of TpoR D1D2 (Chachoua et al., 2016). Finally, the P-domain of CALR del52 was even more accessible to deuterium uptake when in complex with the TpoR D1D2 domain (sD1D2) (FIG. 3G), demonstrating that it is not involved in interaction with the receptors, as previously suggested (Araki et al., 2016).

### Example 3: Comprehensive Model of CALR Del52 Mutant Tail Interaction with TpoR Extracellular Domain

### Materials and Methods

Mutagenesis: All alanine scan mutants were made alternatively using the QuickChange (Agilent), the KLD enzyme mix (NEB) procedure following the manufacturer instruction or purchased from Genscript. All constructs were verified by sequencing.

Transcriptional Dual Luciferase Assay: Transcriptional Dual luciferase assays were performed as described in Chachoua et al., 2016. Briefly, for alanine scanning, HEK293T and y2a were transfected with plasmids coding for STAT5, murine JAK2 and indicated human TpoR mutants (all in pMX-IRES-GFP vector) and either CALR WT, CALR del52 of empty vector (in pMSCV-IRES-mCherry). For other assays, HEK29ET were transfected or human TpoR WT with indicated CALR species. In both cases, SpiLuc reporter was used as a readout of STAT5 transcriptional activation and pRLTK was used as an internal control (Promega). Cells were stimulated, or not, with 25 ng/ml of rhTpo (Milteneyi Biotec) as indicated.

Molecular Dynamics and docking simulations: Sequences of TpoR extracellular and transmembrane regions and of CALR Del52 were profiled for secondary structure, intrinsic disorder and accessibility propensities with state-of-the-art predictors (Barik et al., 2020). Closest templates were retrieved with Phyre 2. Modeller 9.21, Alpha-Fold2 and Rosetta Folding were used to effectively build the 3D models and identify the interaction interface between the two CALR del52 mutants. HDx-MS data was used to identify contacts between TpoR and CALR del52 in the formation of the tetramer complex. The ER specific G3M9 glycans of TpoR in contact to CALR were modeled with Glycopack in the configuration consistent with NMR data while the rest are of complex type, built in agreement with SAGS Database. The HDx-MS identified contacts and the solid-NMR data on the TM region configuration of TpoR dimer were used as constraints in generating the overall 2CALR-2TpoR model. This glycoproteic tetramer was immersed into a full-atom representation of the environment—consisting in a lipid bilayer of 1607 POPC molecules accommodating the TM region of TpoR and in 409966 TIP3P water molecules and 63 Sodium ions describing the solvent region hydrating the rest of the tetramer. This overall system consisting of ˜1.5 million atoms was subjected to a complete molecular dynamics cycle using OpenMM. First the system was minimized using L-BFG algorithm till a threshold of 1 kJ/mole. Heating was then performed slowly over a 13 ns period, from 0 to 300K in an NPT ensemble at 1 bar pressure and a membrane surface tension of 340 bar-nm using a Langevin integrator of 2fs timestep, with hydrogen bonds constrained in order to achieve this high timestep. Production was then continued for 20 ns. Visualization & Analysis was performed using VMD 1.9.3 and in-house Python 3.6 scripts, which use the MDTraj library.

### Results

Interaction sites between CALR del52 mutant tail and TpoR ECD were first probed by performing a complete alanine scanning of the D1D2 domain of full length TpoR by mutating three-four amino acids at a time for most mutants or eight amino acids for three of them. Since the mutant tail is strictly required for activation, but not for binding, which also occurs through glycans, the resulting mutants were probed for their ability to be activated in a STAT5 transcriptional luciferase assay by CALR del52 full length or by the thrombopoietin (Tpo) ligand. Results revealed that the majority of mutants able to inhibit CALR del52 dependent activation of the full-length receptor were located in the D1 domain or at the C-terminus of D2 (data not shown). Inhibitory mutations were particularly dense at the N-terminus of the TpoR and concentrated in the inner/lateral face of the D1 domain.

Given the key role of D1 in the mutant tail binding, the interaction of D1 alanine mutants with CALR del52 P-C domain was further probed and it was found similar results as with full length CALR del52 (data not shown), suggesting they worked by preventing binding or activation mediated by the mutant tail.

In view of the very high proportion of basic residues (Arg) in CALR del52 mutant tail, acidic residues of TpoR which could be involved in interaction with the mutant tail were looked at. One extensive region rich in acidic residues at the very N-terminus of TpoR encompassing residues 37PLKCFSRTFEDLTCFWDEEE56 (SEQ ID NO: 7) appeared potentially critical for TpoR activation. Out of the 62 mutants tested, all the ones inhibiting CALR del52 mediated activation equally prevented Tpo induced activation of the TpoR, while the reverse was not true (data not shown). This suggests that all sites used by Tpo for activation are also used by CALR del52, explaining a decreased Tpo effect on cells co-expressing TpoR and CALR del52 (Marty et al., 2016), but that CALR del52 also employs additional sites not used by the Tpo ligand.

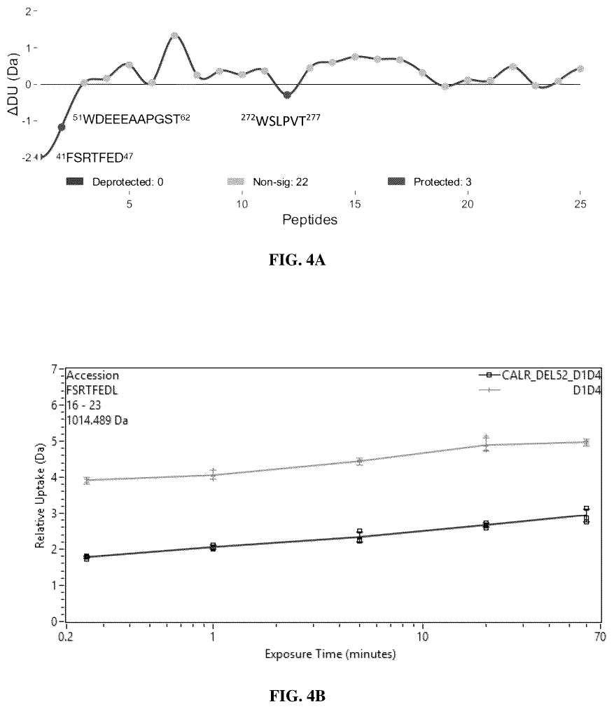

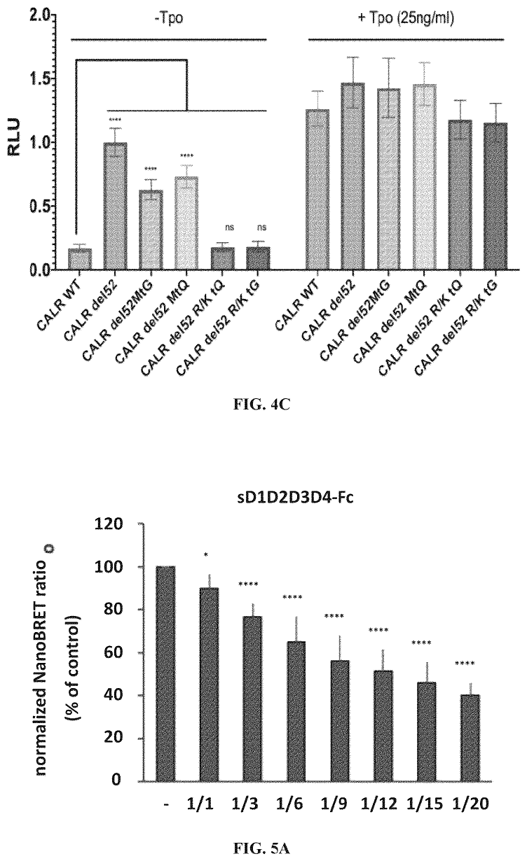

To further explore the binding sites of CALR del52 mutant tail on TpoR ECD, HDx-MS was performed to measure differential deuterium exchange rates between full mature TpoR ECD (labelled D1D4) or CALR del52-D1D4 complex that we showed interacted through the mutant tail as described above (see FIG. 2A). Amongst the covered region of TpoR ECD, three peptides exhibited significant differential deuterium exchange in presence of CALR del52, namely the 41FSRTFED47 peptide (SEQ ID NO: 59), the 51WDEEEAAPGST62 peptide (SEQ ID NO: 61) and the 272WSLPVT277 peptide (SEQ ID NO: 64) (FIG. 4A). In line with the alanine scanning data, the 41FSRTFED47 peptide (SEQ ID NO: 59) containing the 44TFED47 fragment (SEQ ID NO: 60) showed strongest deuterium exchange differential (FIG. 4A-B), followed by the highly negatively charged 51WDEEEAAPGST62 fragment (SEQ ID NO: 61) whose differential deuterium exchange was due to the 51WDEE54 fragment (SEQ ID NO: 62) since another peptide encompassing 55EAAPGST62 (SEQ ID NO: 63) did not show significant exchange differential between the two conditions (not shown). More surprisingly, a significant, albeit lower, exchange differential was also detected with the 272WSLPVT277 peptide (SEQ ID NO: 64) present at the C-terminus of the D2 domain. No other detected peptide showed significant deuterium reduction exchange between the two conditions, although incomplete sequence coverage could not rule out the absence of other interacting sites between the two proteins.

To further explore the interaction of CALR del52 mutant tail with TpoR, molecular dynamics (MD) were used to obtain the structure of the TpoR D1D2 domains. Once the model generated, two complex glycans were attached to glycosylation sites Asn117 and Asn178. In order to assess the model stability, 5 MD simulation runs were performed, each of 1 μs length, in explicit solvent. The final model was in agreement to the one recently published by AlphaFold 2.0. Sequence analysis indicates that TpoR D1D2 has an unbalanced charged composition with an excess of 11 negatively charged acidic amino acids (data not shown). In addition, the model identifies one extensive (S1) and a second more localized (S2) negatively charged region of the TpoR surface. These acidic regions are particularly prone for interaction with the highly basic C-terminus tail of CALR mutant, which was showed to adopt an extensive helical structure up to residue 394. The mutant tail is indeed rich in basic (Lys/Arg) and hydrophobic (Met) residues.

To assess the role of hydrophobic versus acidic residues in activation of the TpoR, either all basic residues (arginine (R) and lysine (K) residues) or all hydrophobic residues (metionine (M) residues) were mutated to Gly (G) or Asn (Q), given CALR del52 R/K tG, CALR del52 R/K tQ, CALR del52 MtG and CALR del52 MtQ, respectively. Results showed that mutations of hydrophobic residues to either Gly or Asn led to decreased activation of the TpoR, quantitatively similar to that observed after modulation of the helix orientation (FIG. 4C, CALR del52 MtG and CALR del52 MtQ). In contrast, mutation of basic residues to either Asn or Gly (CALR del52 R/K tG and CALR del52 R/K tQ) led to complete loss of CALR del52 ability to activate the TpoR (FIG. 4C). These results are in agreement with previous published data (Elf et al., 2016) and suggest that the interaction between the mutant tail and the TpoR ECD relies essentially on basic-acidic interactions.

Then, in silico simulation was performed to probe most stable interacting sites of the mutant tail alone to TpoR D1D2 domains. After modelling of CALR del52 mutant tail, docking trials were performed using three main start configurations of the complex based on the acidic areas of TpoR set as inputs in HADDOCK for TpoR D1D2-CALR del52 mutant tail complex optimizations searches. The top configurations were further optimized using 500 ns Molecular Dynamics runs to obtain 3 final poses in which the last residues of the mutant become unfolded (sata not shown), confirming FTIR data.

The free energy (ΔG) wa further estimated using the Prodigy. There were very large number of microstates of the complex showing very large negative values of the free energy (ΔG<−9 kcal/mol, data not shown), consistent with a high binding affinity of CALR del52 mutant tail for TpoR D1D2. Hence, even the small negative cluster region found in the N-ter region of TpoR-D1, by its own, has a significant affinity for CALR del52 mutant tail. Interestingly, in the process of engaging this TpoR-D1 region, CALR del52 mutant tail was shown by the MD experiments to be able to induce a dislocation and unfolding of the helix found at the N-ter end of TpoR. The computational work indicated that CALR del52 mutant tail has the ability to engage TpoR with very high affinity in a very large number of micro-configurations that target both the continuous acidic area found mainly on D1 (and partly on D2), but also the small acidic patch in the N-terminal region of D1. This “anaconda effect” by which CALR del52 is able to engage its TpoR “pray” in very many ways through both its lectin and mutant tail is mainly due to a synergistic, delocalized effect induced by the charge complementarity, which is extended over very large areas of the two interactors, combined with the significant flexibility of CALR del52 mutant tail, which in this way is able to mold over TpoR more rigid geometry. This suggested that localized interventions (e.g. by point mutations) may only modulate, not completely disrupt the overall interaction between the two molecular objects. The T, E and D residues of the 44TFED47 motif were predicted to directly interact with negatively charged residues on CALR del52 mutant tail by MD simulation by the model labelled as pose 2, in line with alanine scanning and HDX-MS data. Likewise, pose 3 predicted interactions between Arg of CALR del52 mutant tail and the 53EE54 of the 51WDEE54 (SEQ ID NO: 62) but also Q64 of the 64QLLY68 (SEQ ID NO: 65) motifs. Mutations of both motifs to alanine led to loss of CALR del52 induced activation.

MD simulations using only the mutant tail showed that the latter was able to bind the receptor in a large variety of micro-configurations. However, binding of TpoR to full length CALR del52 also involves strong interaction between the N-domain and immature glycans on Asn117. Therefore, AlphaFold 2.0 predictions were used to complete the modelization of TpoR to obtain the full extracellular domain of the receptor which were inserted into POPC lipid bilayers. TpoR monomers were dimerized through their TM domain with residue L508 in the interface as in the active configuration in presence of CALR del52 (FIG. 2F). CALR del52 dimers were docked to dimers of TpoR through glycan binding domains and mutant tail based on experimental data and energy minimization (data not shown). The final structure places the mutant tail in a configuration where main interacting sites are located around the 44TFED47 (SEQ ID NO: 60) motif, in line with the HDx-MS and alanine scanning data described above. Likewise, immature glycans on Asn117 of the receptor interacting with the N-domain pocket containing key residues involved in glycans binding including C105, Y109, D135 and W319.

Molecular Dynamics simulation of 20 ns ware then run on the whole tetramers. The complex remained very stable during this timeframe, with the exception of the very flexible P-domain (video not shown). Contacts between TpoR and CALR molecules were analyzed over the course of the simulation. Most contacts relied on basic-acidic interactions and occurred both in cis and in trans, thereby further stabilizing TpoR dimers.

Therefore, it is proposed herein a model where CALR del52 interacts through two regions with TpoR D1 domain. First, the mutant directly interacts with multiple negatively charged residues on the inner/lateral face of TpoR D1 domain. This interaction between TpoR and CALR del52 is further stabilized by strong interactions between CALR glycan binding domain and immature sugar moieties mainly on Asn117 of TpoR. This anaconda effect of CALR mutant protein binding to two different sites of TpoR explains both its high affinity and specificity for the receptor.

### Example 4: Fec-Coupled TpoR ECD for Binding CALR Mutant

### Materials and Methods

Fusions of the Fc fragment of a huIgG1 or muIgG2B (depending on experiments) to different fragments of the TPOR extracellular domain were performed. Such method was successfully used for the treatment of several pathologies including rheumatoid arthritis which is treated with Etanercept®, a fusion protein between the extracellular domain of the Tumor Necrosis Factor (TNF) and the Fc fragment able to trap and block the pathological effect of the TNF (Zhao et al., 2019; Korth-Bradley J. M. et al, 2000). Three Fc-coupled soluble species of the TPO receptors were developed for the inhibition of CALR mutants binding and activation of the TPOR. These soluble species contain either the entire extracellular domain of the receptor (referred to as D1D2D3D4), the first two domains (referred to as D1D2) or exclusively the first domain of the receptor (referred to as D1). All are coupled to the Fc fragment through the C-terminus. Their mode of action is twofold. First, they will act as trap molecule for circulating CALR mutants and thereby prevent its effect of rogue cytokine (Pecquet et al., 2019). Secondly, they will compete with surface exposed TPOR bound for binding to CALR mutant.

Bioluminescence Energy Transfer (BRET) was used to study the ability of these soluble species to prevent binding of CALR mutant to TPOR (see Example 1). The technique enables to study the interaction between a NanoLuc tag molecule (here, TPOR) and a HaloTag coupled partner (here, CALR del52) directly in living cells. When the NanoLuc and HaloTag are in close proximity (<10 nm), energy transfer between the NanoLuc (donor) and HaloTag (acceptor) leads to a shift in the BRET ratio which is used as a readout for a close proximity between the two partners.

### Results

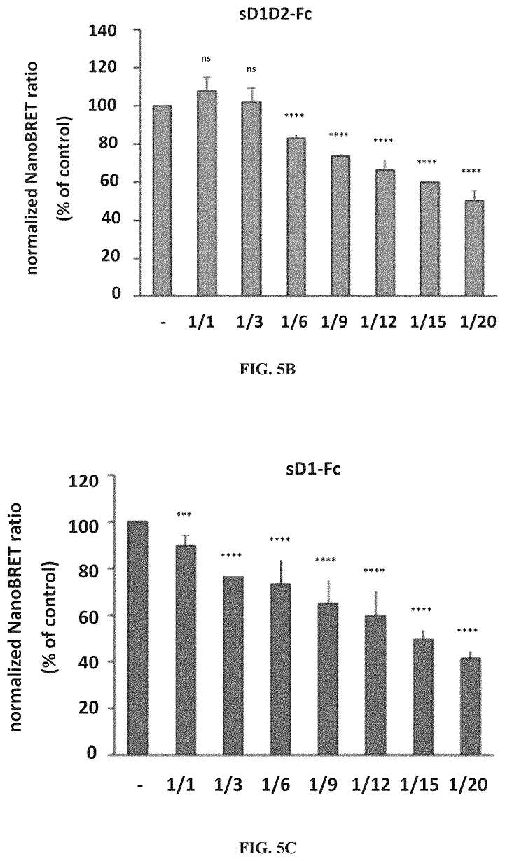

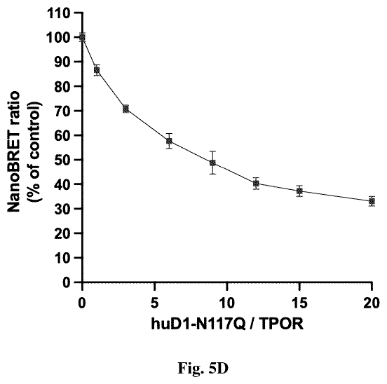

All three soluble fusion proteins, namely Fc-D1, Fc-D1D2 and Fc-D1D2D3D4, are able to inhibit the binding of CALR mutant to full length TPOR (FIG. 5A-C). The fact that the D1 domain alone is able to prevent this binding suggests that interactions between CALR mutant and the receptor could occur exclusively via the D1 domain, even when comprised in a fusion protein (here with Fc fragment of IgG).

Critically, FIG. 5D shows that the inhibition of CALR mutant interaction with TPOR is not dependent on the presence of N-glycosylation at position 117. Thus, while the presence of immature N-glycans is required for complete activation of TPOR by CALR mutant (Chachoua et al., Blood 2016), inhibition of the CALR mutant-TPOR interaction is not reliant on immature N-glycosylation. Specifically, a mutant of D1 that lacks N117 remains able to inhibit the binding of CALR mutant to full length TPOR (FIG. 5D).

### Example 5: TpoR ECD Fragment D1 is Able to Inhibit the Binding of CALR Mutant to TpoR in Trans

### Materials and Methods

Soluble extracellular domain (ECD) species TPOR that are -Fc fused, namely D1-Fc and D1D2D3D4-Fc, were collected after their secretion by cultured HEK293T cells. They were added to the culture medium of hematopoietic cell line Baf3 cells stably expressing human TPOR at their surface together with CALR mutant (Balligand et al., Leukemia 2016). The ability of the Fc-coupled TPOR species to disrupt the interaction between CALR mutant and TPOR was assessed by measuring the level of cell surface CALR mutant in presence of control vehicle or the Fc fragment alone, the D1-Fc or the D1D2D3D4-Fc fragment at the same concentration. The measurement were performed by flow cytometry with an antibody recognizing specifically the mutant CALR at the cell surface of living cells.

### Results

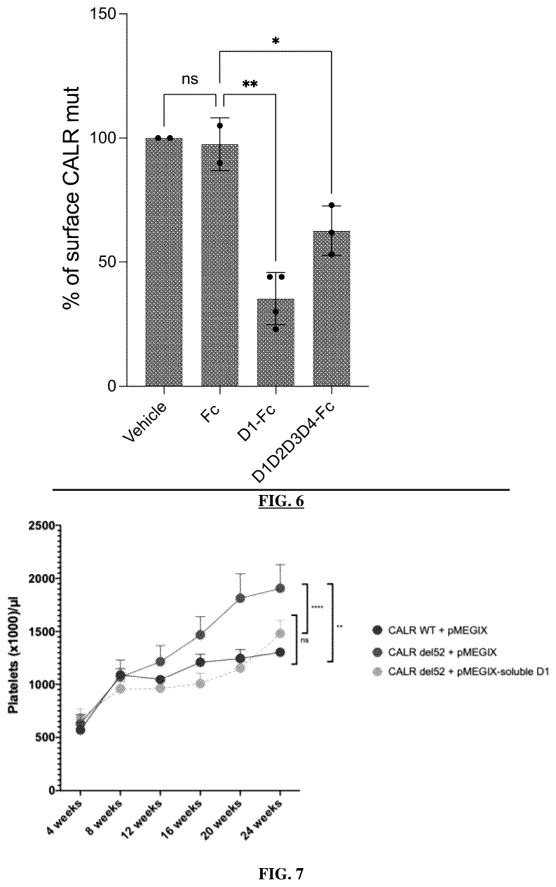

At the concentration tested, D1-Fc fusion protein was able to reduce level of CALR mutant at the cell surface of over 50%. This was specific to the D1-Fc as the Fc control did not modify cell surface levels of CALR mutant (FIG. 6). In comparison to D1-Fc, the D1D2D3D4-Fc had a lower ability to inhibit the interaction between TPOR and CALR with less than 50% inhibition (FIG. 6). These results demonstrate that (i) the D1-Fc is able to inhibit the binding of mutant CALR to TpoR in trans (acting from the extracellular space) and (ii) D1-Fc is a more potent inhibitor that D1D2D3D4-Fc.

### Example 6: In Vivo Application of TpoR ECD Fragment for Treating Essential Thrombocythemia

### Materials and Methods

Next, a mouse model genetically engineered was used to express endogenous levels of CALR mutant. The mouse exhibits a phenotype of Essential Thrombocythemia as described in Balligand et al., 2020. Briefly, small guiding RNAs (sgRNAs) were cloned into the px330 vector (Addgene #42230). The generated vector was microinjected into the nuclei of fertilized B6D2 mouse zygotes. For the creation of the del52 mutation, a single-stranded oligo DNA was co-injected as template to achieve homology-directed repair (HDR) as described in Balligand et al., 2020. Selected pups were crossed with C57BL/6-DBA/2 (B6D22.F1) mice and subsequent generation were selected based on the presence of the del52 mutation in the Calr exon 9 by PCR screening. Bone marrow cells from these mice were isolated and transduced with viral particles containing DNA either of the soluble D1 species (non-coupled to a Fc fragment) or of an empty vector used as control. Recipient mice were lethally irradiated and transplanted with infected cells. The phenotype of these mice was followed over a period of 24 weeks to assess the ability of the soluble D1 species to prevent the development of Essential Thrombocythemia in vivo by measuring the platelets concentration used as a readout of the ET phenotype.

### Results

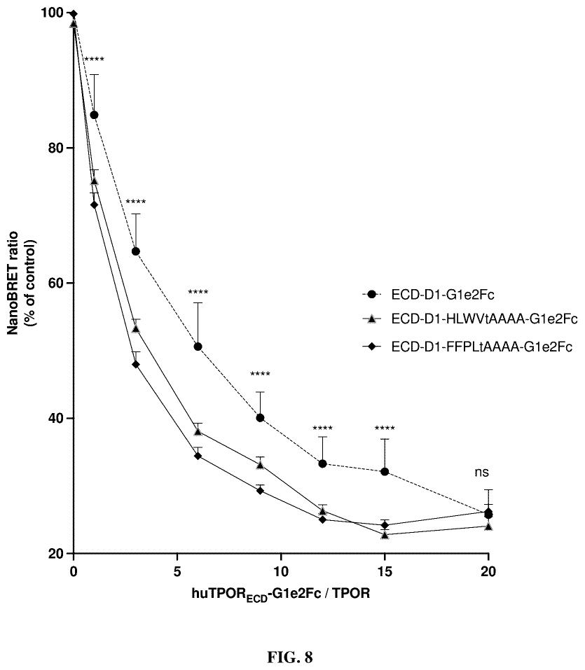

As expected, control mice with ET (CALR del52+pMEGIX) exhibited increasing levels of circulating platelets, reminiscent to the ET phenotype observed in patients (FIG. 7). In sharp contrast, when cells were infected with the soluble D1 domain of TPOR (CALR del52+pMEGIX-soluble D1), they did not develop an ET phenotype and exhibited platelet levels similar to wild-type littermates (CALR WT+pMEGIX) (FIG. 7). This showed that the inhibition of CALR mutant via soluble D1 molecule is able to prevent the development of the disease in vivo.

### Example 7: FFPLHLWV Motif is not Involved in the Interaction

### Materials and Methods

The FFPLtAAAA and HLWVtAAAA mutants of D1 were purchased from Genscript. All constructs were verified by sequencing.

Bioluminescence Energy Transfer (BRET) was used to study the ability of The D1-Fc mutants (“FFPLtAAAA” and “HLWVtAAAA”) to compete with the interaction between full length TpoR and CALR del52 and compare their efficient with the non-mutatent D1-Fc fusion protein (see example 1). The technique enables to study the interaction between a NanoLuc tag molecule (here, TPOR) and a HaloTag coupled partner (here, CALR del52) directly in living cells. When the NanoLuc and HaloTag are in close proximity (<10 nm), energy transfer between the NanoLuc (donor) and HaloTag (acceptor) leads to a shift in the BRET ratio which is used as a readout for a close proximity between the two partners.

FFPLtAAAA mutant of D1 (SEQ ID NO: 87):
QDVSLLASDSEPLKCFSRTFEDLTCFWDEEEAAPSGTYQLLYAYPREKP
RACPLSSQSMPHFGTRYVCQFPDQEEVRLAAAAHLWVKNVFLNQT
HLWVtAAAA mutant of D1 (SEQ ID NO: 88):
QDVSLLASDSEPLKCFSRTFEDLTCFWDEEEAAPSGTYQLLYAYPREKP
RACPLSSQSMPHFGTRYVCQFPDQEEVRLFFPLAAAAKNVFLNQT

### Results

These results demonstrate that the polypeptide comprising D1 can compete TPOR independently of the FFPLHLWV motif. Indeed, mutations of this motif to alanines rather increase D1 ability to disrupt the TPOR-CALR mutant interaction (FIG. 8).

### Example 8: Comprehensive Model of the TPOR-CALR Mutant Complex

### Material and Methods

Templates from AlphaFold2 and Rosetta Folding were used to effectively build the tetrameric 3D models and identify the interaction interface between the two CALR mutants. HDx-MS data was used to identify contacts between TPOR and CALR Del52 in the formation of the tetramer complex. The ER specific G1M9 glycans of TPOR in contact to CALR were modeled with Glycopack 59 in the configuration consistent with NMR data 60 while the rest are of complex type, built in agreement with SAGS Database 61,62. The HDx-MS identified contacts and the solid-NMR data on the TM region configuration of TPOR dimer were used as constraints in generating the overall 2CALR-2TpoR model. This glycoproteic tetramer was immersed into a full-atom representation of the environment—consisting in a lipid bilayer of 1907 POPC molecules accommodating the TM region of TpoR and in 478479 TIP3P water molecules, 1328 chloride and 1402 sodium ions describing the solvent region hydrating the rest of the tetramer using the CHARMM-GUI server 63. This overall system consisting of ˜1 million atoms was subjected to a mild simulated annealing procedure consisting in a start minimization, heating to 300K followed by cooling to OK and final extended minimization, using NAMD v.2.13 64 CHARMM36 forcefield 65-67. The same procedure was used to build TPOR-CALR Ins5 complex. The TpoR-CALR-Del52/ins5 models were further subjected to 3 molecular dynamics runs to explore the configuration sample space.

### Results

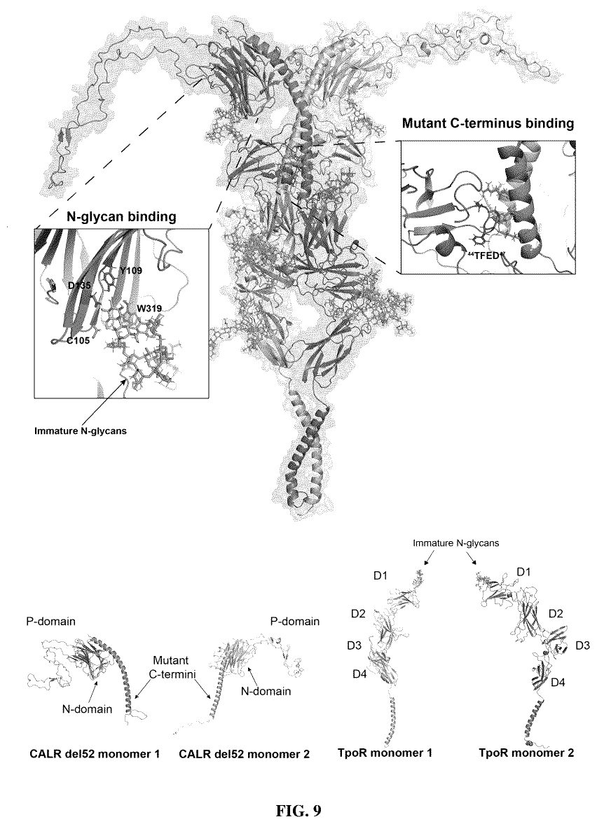

On the basis of our experimental data, an atomistic model of the complete TPOR-CALR mutant tetrameric complex was generated. The data indicated that binding of CALR del52 C-terminus alone to TPOR could occur in a variety of micro-configurations but that the extended Si acidic region centered on 44TFED47 was key for interaction. Yet, binding of TPOR to full length CALR del52 also involves strong interaction between specific residues of the N-domain and immature N-glycans on Asn117. AlphaFold 2.0 was used to complete the modeling of TPOR and generate the full extracellular domain and transmembrane domain of the receptor. TPOR monomers were dimerized through their TM domain with residue L508 in the interface as in the active configuration in presence of CALR del52 (FIG. 2F). CALR del52 dimer was docked to dimer of TPOR taking into consideration experimental data indicating that binding occurs concomitantly between immature N-glycans on Asn117 of TPOR and residues of CALR N-domain and between TPOR S1 acidic region and CALR mutant C-terminus. The final structure places the mutant C-terminus in a configuration where the main interacting sites are located around the 44TFED47 motif, in line with above results. Likewise, immature N-glycans on Asn117 of TpoR interact with the N-domain pocket containing key residues involved in N-glycans binding including C105, Y109 and W319 (FIG. 9). This glycoproteic tetramer was embedded in a POPC lipid bilayer and a water box (a total of ˜1 million atoms) were subjected to triplicate all-atom molecular dynamics simulations for 100 ns. The complex remained stable during this timeframe, except for the very flexible P-domain. Most contacts identified during the simulations relied on basic-acidic interactions and occurred both in cis and in trans, thereby further stabilizing TpoR dimers. They involved the 44TFED47 motif but also other negative patches including 96PDQEE100 and 154WEEP157 of the extended negative patch present in D1 (Table 1).

TABLE 1
Contacts between CALR del52 and TpoR during triplicate 100 ns MD
runs of the CALR del52-TpoR tetrameric complex. Contacts are defined
as residues with a distance <8Ä. Only contacts present
in >60% of frames (average of triplicates) are shown.

| | CALR del52 residue | % of contacts |
|---|---|---|
| R43 | R388 | 68.33 ± 44.78 |
| R43 | M391 | 66.33 ± 46.91 |
| R43 | S392 | 65.67 ± 46.45 |
| R43 | P393 | 65.67 ± 46.45 |
| T44 | R379 | 87.00 ± 14.35 |
| T44 | R382 | 73.00 ± 37.48 |
| T44 | T383 | 85.00 ± 21.21 |
| T44 | R384 | 89.33 ± 15.08 |
| T44 | K386 | 100.00 ± 0.00 |
| T44 | M387 | 66.67 ± 46.44 |
| T44 | R388 | 77.33 ± 32.06 |
| T44 | K390 | 63.00 ± 14.17 |
| T44 | M391 | 84.33 ± 22.16 |
| F45 | R379 | 89.67 ± 11.90 |
| F45 | R382 | 82.67 ± 23.81 |
| F45 | T383 | 68.33 ± 44.78 |
| F45 | K386 | 100.00 ± 0.00 |
| F45 | M391 | 76.67 ± 23.46 |
| E46 | R378 | 76.67 ± 33.00 |
| E46 | R379 | 100.00 ± 0.00 |
| E46 | M380 | 96.33 ± 5.19 |
| E46 | R381 | 67.33 ± 46.20 |
| E46 | R382 | 99.33 ± 0.94 |
| E46 | T383 | 100.00 ± 0.00 |
| E46 | R384 | 96.33 ± 2.87 |
| E46 | K386 | 99.00 ± 1.41 |
| D47 | R379 | 99.00 ± 1.41 |
| D47 | T383 | 73.67 ± 37.24 |
| D47 | R384 | 100.00 ± 0.00 |
| D47 | K386 | 64.00 ± 24.18 |
| D47 | R388 | 71.00 ± 41.01 |
| L48 | R379 | 79.67 ± 28.76 |
| Y63 | D71 | 62.00 ± 43.85 |
| Y63 | A72 | 60.00 ± 43.20 |
| Y63 | V321 | 64.00 ± 44.03 |
| Y63 | K322 | 64.67 ± 36.06 |
| F95 | R379 | 76.00 ± 33.94 |
| P96 | R376 | 62.67 ± 23.46 |
| P96 | R379 | 80.33 ± 23.70 |
| D97 | R372 | 80.00 ± 28.28 |
| D97 | M375 | 74.00 ± 36.77 |
| D97 | R376 | 97.67 ± 1.70 |
| D97 | R379 | 99.67 ± 0.47 |
| Q98 | M371 | 67.67 ± 40.79 |
| Q98 | R372 | 72.67 ± 21.23 |
| Q98 | M375 | 82.33 ± 24.98 |
| Q98 | R376 | 83.00 ± 15.51 |
| Q98 | R379 | 98.33 ± 1.25 |
| Q98 | R382 | 74.33 ± 21.76 |
| E99 | M371 | 61.67 ± 22.94 |
| E99 | R372 | 61.33 ± 43.74 |
| E99 | M375 | 99.33 ± 0.94 |
| E99 | R379 | 92.67 ± 5.79 |
| E100 | R372 | 70.00 ± 21.77 |
| V101 | R379 | 82.67 ± 23.81 |
| R102 | R379 | 70.00 ± 31.19 |
| R102 | R382 | 68.67 ± 23.10 |
| A134 | K390 | 72.67 ± 17.21 |
| P135 | K390 | 81.33 ± 16.82 |
| P136 | R389 | 67.33 ± 33.48 |
| P136 | K390 | 75.33 ± 15.20 |
| S137 | K386 | 63.67 ± 39.53 |
| S137 | M387 | 63.00 ± 40.47 |
| S137 | R388 | 63.67 ± 42.19 |
| S137 | R389 | 98.00 ± 2.83 |
| S137 | K390 | 100.00 ± 0.00 |
| S137 | M391 | 90.33 ± 9.18 |
| S137 | S392 | 90.00 ± 10.80 |
| S137 | A394 | 63.33 ± 40.30 |
| I138 | R388 | 90.67 ± 1.70 |
| I138 | R389 | 99.67 ± 0.47 |
| I138 | K390 | 100.00 ± 0.00 |
| I138 | M391 | 100.00 ± 0.00 |
| I138 | S392 | 100.00 ± 0.00 |
| I138 | P393 | 100.00 ± 0.00 |
| I138 | A394 | 100.00 ± 0.00 |
| I138 | R395 | 98.33 ± 1.25 |
| I138 | R397 | 99.00 ± 0.82 |
| I139 | A394 | 94.00 ± 4.32 |
| K140 | P393 | 93.67 ± 4.92 |
| K140 | A394 | 100.00 ± 0.00 |
| K140 | R395 | 82.00 ± 3.74 |
| K140 | P396 | 64.67 ± 24.23 |
| K140 | R397 | 88.33 ± 8.34 |
| M142 | A394 | 65.67 ± 39.08 |
| M142 | R395 | 73.33 ± 34.24 |
| M142 | P396 | 80.67 ± 25.25 |
| M142 | R397 | 80.00 ± 26.87 |
| Q151 | P396 | 77.00 ± 25.15 |
| Q151 | R397 | 81.67 ± 25.93 |
| Q151 | T398 | 82.33 ± 22.22 |
| Q151 | S399 | 70.33 ± 35.78 |
| I152 | R397 | 100.00 ± 0.00 |
| S153 | A394 | 99.00 ± 1.41 |
| S153 | R395 | 100.00 ± 0.00 |
| S153 | P396 | 91.00 ± 12.73 |
| S153 | R397 | 100.00 ± 0.00 |
| S153 | T398 | 68.67 ± 22.22 |
| W154 | R389 | 62.67 ± 25.53 |
| W154 | A394 | 99.33 ± 0.94 |
| W154 | R395 | 100.00 ± 0.00 |
| W154 | P396 | 73.67 ± 13.60 |
| W154 | R397 | 100.00 ± 0.00 |
| E155 | R388 | 94.67 ± 0.47 |
| E155 | R389 | 100.00 ± 0.00 |
| E155 | K390 | 99.67 ± 0.47 |
| E155 | M391 | 72.00 ± 32.57 |
| E155 | S392 | 100.00 ± 0.00 |
| E155 | P393 | 100.00 ± 0.00 |
| E155 | A394 | 100.00 ± 0.00 |
| E155 | R395 | 100.00 ± 0.00 |
| E155 | P396 | 100.00 ± 0.00 |
| E155 | R397 | 100.00 ± 0.00 |
| E155 | C400 | 71.67 ± 37.28 |
| E155 | R401 | 64.33 ± 42.68 |
| E155 | E402 | 72.00 ± 33.98 |
| E155 | A403 | 89.00 ± 9.63 |
| E156 | R385 | 70.00 ± 40.31 |
| E156 | K386 | 99.67 ± 0.47 |
| E156 | M387 | 74.67 ± 6.65 |
| E156 | R388 | 94.67 ± 2.05 |
| E156 | R389 | 100.00 ± 0.00 |
| E156 | K390 | 100.00 ± 0.00 |
| E156 | S392 | 73.67 ± 37.24 |
| E156 | R395 | 100.00 ± 0.00 |
| E156 | R397 | 100.00 ± 0.00 |
| E156 | C400 | 66.00 ± 44.59 |
| E156 | R401 | 65.00 ± 44.64 |
| P157 | K386 | 100.00 ± 0.00 |
| P157 | R389 | 100.00 ± 0.00 |
| P157 | K390 | 94.33 ± 7.32 |
| P157 | R397 | 73.67 ± 14.06 |
| A158 | R382 | 80.33 ± 20.07 |
| A158 | T383 | 78.67 ± 5.73 |
| A158 | R385 | 100.00 ± 0.00 |
| A158 | K386 | 100.00 ± 0.00 |
| A158 | M387 | 99.33 ± 0.47 |
| A158 | R388 | 99.67 ± 0.47 |
| A158 | R389 | 100.00 ± 0.00 |
| A158 | K390 | 99.67 ± 0.47 |
| A158 | M391 | 60.67 ± 43.39 |
| P159 | R382 | 88.67 ± 10.40 |
| P159 | R385 | 97.00 ± 0.82 |
| P159 | K386 | 100.00 ± 0.00 |
| P159 | R389 | 91.00 ± 8.83 |
| E160 | R382 | 99.67 ± 0.47 |
| E160 | T383 | 63.67 ± 15.17 |
| E160 | R385 | 79.67 ± 10.21 |
| E160 | K386 | 100.00 ± 0.00 |
| I161 | K386 | 89.67 ± 6.85 |
| S162 | R385 | 77.00 ± 21.23 |
| S162 | R389 | 71.00 ± 38.91 |
| Y167 | R395 | 60.67 ± 32.07 |
| D175 | W408 | 81.33 ± 24.31 |
| P176 | W408 | 77.67 ± 29.47 |
| S232 | S399 | 61.00 ± 35.05 |
| S232 | E402 | 70.33 ± 41.96 |
| A233 | S399 | 60.67 ± 34.50 |
| A233 | E402 | 64.00 ± 41.41 |
| L234 | R397 | 100.00 ± 0.00 |
| L234 | T398 | 95.33 ± 4.64 |
| L234 | S399 | 96.00 ± 4.32 |
| L234 | C400 | 80.33 ± 27.11 |
| L234 | R401 | 81.67 ± 22.48 |
| L234 | E402 | 69.67 ± 41.49 |
| T235 | R397 | 99.33 ± 0.94 |
| T235 | T398 | 67.67 ± 45.73 |
| A236 | R395 | 98.67 ± 1.89 |
| A236 | P396 | 97.33 ± 3.77 |
| A236 | R397 | 100.00 ± 0.00 |
| A236 | T398 | 99.67 ± 0.47 |
| A236 | S399 | 97.67 ± 3.30 |
| A236 | C400 | 66.67 ± 47.14 |
| E237 | R389 | 89.33 ± 15.08 |
| E237 | R395 | 100.00 ± 0.00 |
| E237 | P396 | 100.00 ± 0.00 |
| E237 | R397 | 100.00 ± 0.00 |
| E237 | T398 | 100.00 ± 0.00 |
| E237 | S399 | 100.00 ± 0.00 |
| E237 | C400 | 99.67 ± 0.47 |
| E237 | R401 | 74.33 ± 35.59 |
| E237 | E402 | 66.67 ± 45.73 |
| G238 | R389 | 92.00 ± 9.93 |
| G238 | R395 | 100.00 ± 0.00 |
| G238 | P396 | 100.00 ± 0.00 |
| G238 | R397 | 100.00 ± 0.00 |
| G238 | T398 | 99.33 ± 0.94 |
| G238 | S399 | 61.00 ± 17.68 |
| G238 | C400 | 67.33 ± 42.71 |
| G239 | R395 | 100.00 ± 0.00 |
| G239 | P396 | 93.67 ± 2.87 |
| G239 | R397 | 100.00 ± 0.00 |
| S240 | P396 | 69.67 ± 21.48 |
| S240 | R397 | 100.00 ± 0.00 |
| S240 | T398 | 87.00 ± 9.27 |
| S240 | S399 | 78.33 ± 27.18 |
| S240 | E402 | 60.33 ± 42.46 |
| C241 | R397 | 100.00 ± 0.00 |
| L242 | T398 | 63.33 ± 29.80 |
| L242 | E402 | 67.00 ± 46.67 |
| W253 | W408 | 82.67 ± 24.51 |
| W253 | T409 | 67.00 ± 24.12 |
| Q255 | W408 | 78.67 ± 30.17 |
| W272 | W408 | 66.00 ± 46.68 |
| S273 | G407 | 60.67 ± 43.52 |
| S273 | W408 | 75.33 ± 34.18 |
| L274 | C404 | 75.00 ± 14.99 |
| L274 | L405 | 75.33 ± 30.07 |
| L274 | Q406 | 85.67 ± 19.57 |
| L274 | G407 | 69.00 ± 43.84 |
| L274 | W408 | 81.67 ± 25.93 |
| L274 | T409 | 62.33 ± 44.39 |
| P275 | Q406 | 77.67 ± 15.63 |
| P275 | G407 | 77.67 ± 29.49 |
| P275 | W408 | 82.67 ± 24.51 |
| P275 | T409 | 72.67 ± 38.66 |

To assess whether this model was also compatible with CALR Ins5 (CALR type 2 mutant), which harbors a similar but longer C-terminus, the CALR Ins5-TpoR tetrameric complex was also generated following the same procedure as for CALR del52 and subjected the complex to all atom molecular dynamics simulations in triplicate. Like for CALR del52, the complex remained stable over the 100 ns timeframe. Analysis of interacting residues over the simulation timeframe revealed that the 44TFED47 motif, 96PDQEE100 motif were conserved in the CALR-Ins5-TpoR tetrameric complex in addition to the 26QDV28 motif (Table 2).

TABLE 2
Contacts between CALR Ins5 and TpoR during triplicate 100 ns MD
runs of the CALR Ins5-TpoR tetrameric complex. Contacts are defined
as residues with a distance <8Ä. Only contacts
present in >60% of frames (average of triplicates) are shown.

| | CALR Ins5 residue | % of contacts |
|---|---|---|
| S25 | S411 | 64.00 ± 45.28 |
| D27 | K409 | 65.33 ± 46.20 |
| D27 | M410 | 66.67 ± 47.14 |
| D27 | S411 | 66.67 ± 47.14 |
| D27 | P415 | 63.67 ± 45.17 |
| D27 | C419 | 60.33 ± 42.99 |
| V28 | M410 | 66.00 ± 46.68 |
| V28 | S411 | 66.67 ± 47.14 |
| V28 | P412 | 66.67 ± 47.14 |
| V28 | A413 | 66.67 ± 47.14 |
| V28 | R414 | 66.67 ± 47.14 |
| V28 | P415 | 66.67 ± 47.14 |
| V28 | R416 | 66.67 ± 47.14 |
| V28 | C419 | 65.00 ± 46.01 |
| S29 | R414 | 60.33 ± 42.82 |
| S29 | P415 | 66.67 ± 47.14 |
| S29 | R416 | 65.67 ± 46.45 |
| S29 | R420 | 65.67 ± 39.72 |
| S29 | E421 | 65.67 ± 46.44 |
| S29 | C423 | 61.33 ± 43.86 |
| L30 | P415 | 64.67 ± 45.79 |
| L31 | M410 | 66.33 ± 46.91 |
| L31 | S411 | 66.00 ± 46.68 |
| L31 | A413 | 65.33 ± 46.23 |
| L31 | R414 | 63.67 ± 45.17 |
| L31 | P415 | 66.67 ± 47.14 |
| L31 | R416 | 62.33 ± 44.39 |
| L31 | S418 | 61.67 ± 44.03 |
| L31 | C419 | 66.67 ± 47.14 |
| A32 | R414 | 66.67 ± 47.14 |
| A32 | P415 | 66.67 ± 47.14 |
| A32 | R416 | 66.67 ± 47.14 |
| A32 | T417 | 65.33 ± 46.23 |
| A32 | S418 | 64.67 ± 45.11 |
| A32 | C419 | 73.67 ± 36.54 |
| A32 | E421 | 81.00 ± 8.64 |
| S33 | P415 | 66.67 ± 47.14 |
| S33 | R416 | 65.00 ± 46.01 |
| S33 | T417 | 60.00 ± 43.20 |
| S33 | S418 | 63.00 ± 44.77 |
| S33 | R420 | 68.00 ± 45.25 |
| F41 | M410 | 66.67 ± 47.14 |
| R43 | K405 | 89.33 ± 10.14 |
| R43 | R408 | 65.33 ± 46.23 |
| R43 | K409 | 66.67 ± 47.14 |
| R43 | M410 | 66.67 ± 47.14 |
| R43 | S411 | 65.00 ± 45.98 |
| T44 | R401 | 97.33 ± 3.09 |
| T44 | R401 | 91.67 ± 7.93 |
| T44 | T402 | 75.67 ± 33.71 |
| T44 | R404 | 77.00 ± 32.53 |
| T44 | R404 | 74.00 ± 16.57 |
| T44 | K405 | 100.00 ± 0.00 |
| T44 | M406 | 81.33 ± 22.29 |
| F45 | R397 | 82.67 ± 16.76 |
| F45 | R398 | 95.33 ± 6.60 |
| F45 | R401 | 75.00 ± 18.24 |
| F45 | R401 | 66.67 ± 47.14 |
| F45 | R404 | 78.00 ± 11.31 |
| F45 | K405 | 97.67 ± 3.30 |
| E46 | M394 | 67.00 ± 28.89 |
| E46 | R397 | 94.67 ± 7.54 |
| E46 | R397 | 65.33 ± 32.43 |
| E46 | R398 | 98.33 ± 1.25 |
| E46 | R398 | 87.33 ± 17.91 |
| E46 | R401 | 100.00 ± 0.00 |
| E46 | R401 | 74.00 ± 36.77 |
| E46 | T402 | 98.00 ± 2.83 |
| E46 | R404 | 69.67 ± 32.25 |
| E46 | R404 | 62.67 ± 17.56 |
| E46 | K405 | 100.00 ± 0.00 |
| D47 | R397 | 62.33 ± 43.74 |
| D47 | R398 | 97.33 ± 3.77 |
| D47 | R398 | 78.67 ± 16.44 |
| D47 | R401 | 88.67 ± 16.03 |
| D47 | T402 | 67.67 ± 40.88 |
| D47 | K405 | 93.33 ± 4.92 |
| L48 | R398 | 78.67 ± 29.47 |
| T49 | M410 | 60.33 ± 42.99 |
| F51 | M410 | 66.67 ± 47.14 |
| D53 | P415 | 63.33 ± 44.97 |
| E55 | R414 | 60.67 ± 43.52 |
| M84 | S411 | 65.67 ± 46.45 |
| M84 | P412 | 66.67 ± 47.14 |
| M84 | A413 | 66.00 ± 46.67 |
| H86 | R414 | 66.67 ± 47.14 |
| F87 | S411 | 64.00 ± 45.37 |
| F87 | P412 | 66.67 ± 47.14 |
| F87 | A413 | 66.67 ± 47.14 |
| F87 | R414 | 66.67 ± 47.14 |
| G88 | R414 | 64.67 ± 45.76 |
| R90 | M410 | 66.67 ± 47.14 |
| R90 | S411 | 66.67 ± 47.14 |
| R90 | P412 | 66.67 ± 47.14 |
| R90 | A413 | 66.67 ± 47.14 |
| R90 | R414 | 65.67 ± 46.45 |
| R90 | P415 | 68.67 ± 42.91 |
| F95 | R398 | 74.00 ± 34.67 |
| F95 | R398 | 63.67 ± 34.76 |
| P96 | R395 | 70.67 ± 40.78 |
| P96 | R398 | 77.33 ± 23.92 |
| P96 | R398 | 64.67 ± 29.41 |
| D97 | R387 | 62.67 ± 43.28 |
| D97 | R391 | 72.33 ± 39.13 |
| D97 | T392 | 63.67 ± 43.18 |
| D97 | M394 | 78.00 ± 31.11 |
| D97 | M394 | 66.67 ± 47.14 |
| D97 | R395 | 86.67 ± 18.86 |
| D97 | R395 | 75.67 ± 28.29 |
| D97 | R398 | 100.00 ± 0.00 |
| D97 | R398 | 94.00 ± 8.49 |
| Q98 | M394 | 99.00 ± 1.41 |
| Q98 | R395 | 68.67 ± 43.61 |
| Q98 | R398 | 100.00 ± 0.00 |
| Q98 | R398 | 99.67 ± 0.47 |
| E99 | M390 | 67.00 ± 30.47 |
| E99 | R391 | 95.33 ± 5.91 |
| E99 | M394 | 100.00 ± 0.00 |
| E99 | M394 | 83.33 ± 22.17 |
| E99 | R395 | 98.33 ± 1.70 |
| E99 | R395 | 73.67 ± 19.60 |
| E99 | R397 | 64.67 ± 34.99 |
| E99 | R398 | 99.67 ± 0.47 |
| E99 | R398 | 93.00 ± 5.72 |
| E99 | R401 | 60.67 ± 34.99 |
| E100 | M394 | 99.00 ± 1.41 |
| E100 | R398 | 80.33 ± 27.81 |
| E100 | R398 | 66.67 ± 23.58 |
| V101 | M394 | 100.00 ± 0.00 |
| V101 | R397 | 60.00 ± 42.59 |
| V101 | R398 | 81.00 ± 26.87 |
| V101 | R398 | 72.33 ± 7.72 |
| V101 | R401 | 63.33 ± 27.72 |
| R102 | M390 | 67.67 ± 45.73 |
| R102 | M394 | 68.00 ± 40.41 |
| R102 | R398 | 67.33 ± 46.20 |
| R102 | R401 | 65.67 ± 24.36 |
| L103 | R397 | 82.00 ± 22.69 |
| L103 | R398 | 65.33 ± 46.20 |
| L103 | R401 | 62.67 ± 37.38 |
| L132 | M410 | 65.33 ± 46.23 |
| A134 | M410 | 63.67 ± 45.11 |
| A134 | S411 | 62.00 ± 10.61 |
| P135 | M410 | 66.00 ± 46.67 |
| P135 | S411 | 91.00 ± 12.73 |
| P135 | P412 | 79.33 ± 17.33 |
| P135 | A413 | 69.33 ± 23.47 |
| P136 | R407 | 89.33 ± 11.73 |
| P136 | P412 | 60.67 ± 42.97 |
| S137 | R407 | 95.67 ± 5.44 |
| S137 | K409 | 84.00 ± 21.92 |
| S137 | M410 | 65.33 ± 32.15 |
| I138 | R407 | 81.33 ± 25.00 |
| I138 | K409 | 66.67 ± 18.12 |
| I138 | S411 | 66.67 ± 23.30 |
| I138 | P412 | 65.67 ± 46.44 |
| I138 | R414 | 62.67 ± 44.58 |
| K140 | P412 | 60.33 ± 19.57 |
| E155 | R407 | 79.33 ± 27.82 |
| E155 | K409 | 99.33 ± 0.47 |
| E155 | M410 | 71.33 ± 38.44 |
| E155 | S411 | 99.67 ± 0.47 |
| E155 | P412 | 88.67 ± 10.34 |
| E155 | R414 | 66.00 ± 46.68 |
| E156 | M406 | 80.00 ± 17.68 |
| E156 | R407 | 100.00 ± 0.00 |
| E156 | R408 | 95.00 ± 7.07 |
| E156 | K409 | 99.67 ± 0.47 |
| E156 | M410 | 68.00 ± 24.06 |
| P157 | K405 | 98.00 ± 1.41 |
| P157 | M406 | 93.67 ± 4.11 |
| P157 | R407 | 100.00 ± 0.00 |
| P157 | K409 | 60.67 ± 22.66 |
| A158 | R404 | 93.00 ± 5.35 |
| A158 | K405 | 100.00 ± 0.00 |
| A158 | M406 | 100.00 ± 0.00 |
| A158 | R407 | 100.00 ± 0.00 |
| A158 | R408 | 91.33 ± 11.56 |
| P159 | R403 | 94.00 ± 3.56 |
| P159 | R404 | 100.00 ± 0.00 |
| P159 | K405 | 100.00 ± 0.00 |
| P159 | K405 | 68.33 ± 43.38 |
| P159 | M406 | 100.00 ± 0.00 |
| P159 | R407 | 100.00 ± 0.00 |
| P159 | R408 | 100.00 ± 0.00 |
| P159 | R408 | 66.33 ± 46.91 |
| P159 | K409 | 98.33 ± 1.70 |
| P159 | K409 | 64.33 ± 22.17 |
| E160 | R400 | 65.67 ± 46.45 |
| E160 | R401 | 95.00 ± 3.27 |
| E160 | R403 | 74.67 ± 10.96 |
| E160 | R403 | 67.33 ± 32.83 |
| E160 | R404 | 100.00 ± 0.00 |
| E160 | R404 | 78.67 ± 29.47 |
| E160 | K405 | 100.00 ± 0.00 |
| E160 | K405 | 78.67 ± 30.17 |
| E160 | M406 | 100.00 ± 0.00 |
| E160 | M406 | 67.00 ± 30.24 |
| E160 | R407 | 77.67 ± 10.50 |
| E160 | R408 | 91.33 ± 11.56 |
| I161 | R404 | 64.67 ± 28.29 |
| I161 | K405 | 95.00 ± 3.74 |
| I161 | K405 | 61.00 ± 40.60 |
| R174 | A430 | 61.00 ± 43.69 |
| D175 | Q425 | 86.33 ± 19.33 |
| D175 | A430 | 64.33 ± 41.59 |
| P176 | L424 | 71.67 ± 38.66 |
| P176 | Q425 | 94.00 ± 8.49 |
| P176 | G426 | 100.00 ± 0.00 |
| P176 | W427 | 84.67 ± 17.52 |
| P176 | T428 | 99.00 ± 1.41 |
| P176 | E429 | 91.33 ± 7.59 |
| P176 | A430 | 65.67 ± 41.02 |
| K177 | L424 | 61.33 ± 34.62 |
| K177 | Q425 | 92.33 ± 10.84 |
| K177 | G426 | 100.00 ± 0.00 |
| K177 | G426 | 63.33 ± 43.56 |
| K177 | W427 | 100.00 ± 0.00 |
| K177 | T428 | 100.00 ± 0.00 |
| K177 | E429 | 99.00 ± 1.41 |
| K177 | E429 | 63.00 ± 44.77 |
| K177 | A430 | 96.00 ± 5.66 |
| N178 | Q425 | 90.67 ± 13.20 |
| N178 | G426 | 100.00 ± 0.00 |
| N178 | G426 | 64.33 ± 43.38 |
| N178 | W427 | 100.00 ± 0.00 |
| N178 | W427 | 64.33 ± 43.56 |
| N178 | T428 | 100.00 ± 0.00 |
| N178 | T428 | 62.67 ± 43.93 |
| N178 | E429 | 99.00 ± 1.41 |
| N178 | E429 | 61.67 ± 42.63 |
| N178 | A430 | 99.00 ± 1.41 |
| S179 | Q425 | 61.00 ± 35.56 |
| S179 | G426 | 98.00 ± 2.83 |
| S179 | W427 | 96.33 ± 5.19 |
| S179 | T428 | 96.33 ± 5.19 |
| S179 | E429 | 96.67 ± 4.71 |
| S179 | A430 | 95.33 ± 3.40 |
| T180 | T428 | 86.33 ± 18.62 |
| T180 | E429 | 86.00 ± 18.40 |
| G181 | Q425 | 69.00 ± 43.84 |
| G181 | G426 | 91.67 ± 8.50 |
| G181 | T428 | 72.67 ± 10.08 |
| P182 | A422 | 63.00 ± 44.77 |
| P182 | L424 | 66.67 ± 47.14 |
| P182 | Q425 | 67.67 ± 45.73 |
| P182 | G426 | 78.67 ± 21.64 |
| E237 | R408 | 60.67 ± 43.52 |
| G238 | K409 | 62.67 ± 43.02 |
| W253 | R416 | 65.00 ± 29.88 |
| Q255 | R416 | 89.33 ± 7.93 |
| W269 | P415 | 83.00 ± 14.97 |
| W269 | R416 | 69.67 ± 32.87 |
| W269 | T417 | 76.00 ± 29.13 |
| W269 | S418 | 85.67 ± 14.70 |
| W269 | S418 | 68.67 ± 20.95 |
| W269 | C419 | 98.67 ± 1.25 |
| W269 | C419 | 68.67 ± 38.13 |
| W269 | R420 | 74.67 ± 14.34 |
| W269 | R420 | 63.00 ± 44.70 |
| G270 | P415 | 77.67 ± 16.13 |
| S271 | P415 | 61.33 ± 31.90 |
| W272 | S411 | 93.67 ± 5.79 |
| W272 | P412 | 85.67 ± 16.78 |
| W272 | A413 | 99.67 ± 0.47 |
| W272 | R414 | 99.67 ± 0.47 |
| W272 | P415 | 100.00 ± 0.00 |
| W272 | R416 | 100.00 ± 0.00 |
| W272 | T417 | 84.33 ± 19.40 |
| W272 | S418 | 79.00 ± 14.72 |
| W272 | C419 | 67.67 ± 38.73 |
| S273 | A413 | 73.00 ± 28.25 |
| S273 | R414 | 72.33 ± 35.61 |
| S273 | R416 | 95.67 ± 5.44 |
| L274 | S411 | 75.67 ± 32.29 |
| L274 | P412 | 98.67 ± 1.25 |
| L274 | A413 | 100.00 ± 0.00 |
| L274 | R414 | 100.00 ± 0.00 |
| L274 | R416 | 90.00 ± 3.56 |
| P275 | A413 | 76.33 ± 26.60 |
| P275 | R414 | 67.67 ± 24.42 |
| P275 | R416 | 94.00 ± 7.12 |

These experimental data and atomistic simulations indicate that CALR mutants interact through two regions of TPOR essentially on the D1 domain. The mutant C-terminus directly interacts with multiple negatively charged residues on the inner/lateral face of TPOR D1 domain represented by the S1 negative patch.

### Example 9: Identification of Residues from D1 Involved in the Binding of TPOR to CALR

### Material and Methods

See Example 4 for the NanoBRET methods.

### Results

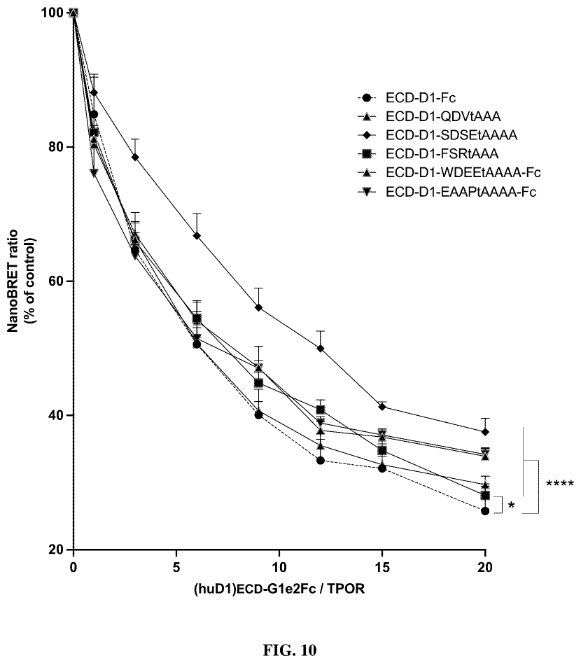

FIG. 10 illustrates NanoBRET signal used as a surrogate for binding between TPOR full length and CALR del52 in presence of increasing amount of human D1 WT and mutants. These data show that charged residues of TPOR, including those in the motifs QDV, SDSE, WDFE, EAAP and FSR are essential for maximal ability of D1 to act as a competitor of the CALR mutant-TPOR interaction. Key residues also identified via HDx-MS and/or Molecular Dynamics simulations (see Example 8) are underlined.

These results correlate with (i) HDx-MS result showing interaction between CALR mutant C-terminus and negative patches on TpoR D1 domain and (ii) Molecular Dynamic simulations identifying additional negatively charged residues as key interactor with mutant CALR. The loss of such residues lead to reduced ability of D1 to act as a competitor of the CALR mutant-TpoR interaction.

1-15. (canceled)

16. A polypeptide comprising an amino acid sequence having at least 75% sequence identity with
(SEQ ID NO: 66)
PLKCFSX₂TFX₁X₁LTCFWX₁X₁X₁X₁AAPSGTYQLLYAYPREKPRACPLS
SQSMPHFGTRYVCQFPX₁QX₁X₁VX₂,
wherein X₁ is D (Asp, Aspartic acid) or E (Glu, Glutamic acid), wherein X₂ is R (Arg, Arginine) or K (Lys, Lysine), and wherein said amino acid sequence of said polypeptide does not comprise SEQ ID NO: 5.

17. The polypeptide according to claim 16, wherein said amino acid sequence has at least 75% sequence identity with SEQ ID NO: 2, SEQ ID NO: 3 or SEQ ID NO: 4.

18. The polypeptide according to claim 16, wherein said polypeptide binds to mutants of calreticulin (CALR) having a positively charged amino acid sequence in the C-terminus tail.

19. The polypeptide according to claim 16, wherein said amino acid sequence comprises SEQ ID NO: 7 and/or at least an amino acid Asn at position 117, wherein said position is defined with respect to the amino acid sequence SEQ ID NO: 1.

20. The polypeptide according to claim 19, wherein said amino acid Asn at position 117 is not glycosylated.

21. A fusion protein comprising a polypeptide according to claim 16.

22. The fusion protein according to claim 21, further comprising a second polypeptide that increases stability and/or decreases immunogenicity of the first polypeptide.

23. The fusion protein according to claim 22, wherein said second polypeptide is a Fc region of an immunoglobulin or a functional equivalent thereof.

24. The fusion protein according to claim 23, wherein said second polypeptide is selected from the group consisting of IgG, IgA, IgD, IgE or IgM.

25. A nucleic acid comprising a sequence encoding a polypeptide according to claim 16 or a fusion protein comprising said polypeptide.

26. A vector comprising a nucleic acid according to claim 25.

27. A pharmaceutical composition comprising (i) a polypeptide according to claim 16 or a fusion protein comprising said polypeptide, or a nucleic acid comprising a sequence encoding for said polypeptide, or a vector comprising said nucleic acid, and (ii) at least one pharmaceutically acceptable vehicle.

28. A method for treating and/or preventing a disease in a subject in need thereof comprising administering to said subject a therapeutically effective amount of:
the polypeptide according to claim 16,
a fusion protein comprising said polypeptide,
a nucleic acid comprising a sequence coding for said polypeptide,
a vector comprising said nucleic acid, or
a pharmaceutical composition comprising (i) said polypeptide, or said fusion protein, or said nucleic acid, or said vector and (ii) at least one pharmaceutically acceptable vehicle.

29. The method according to claim 28, wherein said disease is a blood cancer.

30. The method according to claim 29, wherein said blood cancer is a myeloproliferative neoplasm (MPN).

31. The method according to claim 30, wherein the MPN is induced by one or more mutations in the gene encoding calreticulin (CALR), in a subject in need thereof.

32. The method according to claim 31, wherein said one or more mutations result in the generation of a positively charged amino acid sequence in the C terminus of CALR.

33. A method for inhibiting the expansion of cancer cells in an individual in need thereof and/or improving the overall survival of an individual having cancer, comprising at least the step of administering to the individual a therapeutically effective amount of:
the polypeptide according to claim 16,
a fusion protein comprising said polypeptide,
a nucleic acid comprising a sequence coding for said polypeptide,
a vector comprising said nucleic acid, or
a pharmaceutical composition comprising (i) said polypeptide, or said fusion protein, or said nucleic acid, or said vector and (ii) at least one pharmaceutically acceptable vehicle.

34. A method for improving the prognostic of an individual having cancer, comprising at least the step of administering to the individual a therapeutically effective amount of:
the polypeptide according to claim 16,
a fusion protein comprising said polypeptide,
a nucleic acid comprising a sequence coding for said polypeptide,
a vector comprising said nucleic acid, or
a pharmaceutical composition comprising (i) said polypeptide, or said fusion protein, or said nucleic acid, or said vector and (ii) at least one pharmaceutically acceptable vehicle.

35. A kit for treating and/or preventing myeloproliferative neoplasms (MPN) comprising (i) a polypeptide according to claim 16, a fusion protein comprising said polypeptide, a nucleic acid comprising a sequence coding for said polypeptide, a vector comprising said nucleic acid or pharmaceutical composition comprising said polypeptide, or said fusion protein, or said nucleic acid and at least one pharmaceutically acceptable vehicle, (ii) means to administer said polypeptide, fusion protein or pharmaceutical composition, and optionally (iii) a further anticancer agent or vaccine.
